(19) United States
(12) Patent Application Publication
(10) Pub. No.: US 2021/0214412 A1
(43) Pub. Date: Jul. 15, 2021

(54) GLUCOSE-RESPONSIVE INSULIN

(71) Applicant: University of Utah Research Foundation, Salt Lake City, UT (US)

(72) Inventor: Danny Hung-Chieh Chou, Salt Lake City, UT (US)

(21) Appl. No.: 17/040,973

(22) PCT Filed: Apr. 15, 2019
(86) PCT No.: PCT/US2019/027484

§ 371 Date: Sep. 23, 2020

Related U.S. Application Data
(60) Provisional application No. 62/658,372, filed on Apr. 16, 2018.

(51) Int. Cl. C07K 14/62; A61P 3/10

(52) U.S. Cl. CPC C07K 14/62; A61P 3/10

(57) ABSTRACT

The present disclosure is concerned with insulin-based peptides, methods of making the peptides, and methods of treating diabetes using these peptides. This abstract is intended as a scanning tool for purposes of searching in the particular art and is not intended to be limiting of the present invention.

### CROSS-REFERENCE TO RELATED APPLICATIONS

This application claims the benefit of U.S. Application No. 62/658,372, filed on Apr. 16, 2018, which is incorporated herein by reference in its entirety.

### REFERENCE TO SEQUENCE LISTING

The Sequence Listing submitted Apr. 15, 2019 as a text file named “21101_0369P1_ST25.txt,” created on Apr. 15, 2019, and having a size of 3,080 bytes is hereby incorporated by reference pursuant to 37 C.F.R. § 1.52(e)(5).

### BACKGROUND

Since the discovery of insulin nearly a century ago, many advancements in insulin design have allowed people with diabetes to improve their glycemic control; however, the risk of hypoglycemia is still the major barrier for tight glycemic control (Brownlee and Hirsch (2006) JAMA: The Journal of the American Medical Association 295(14): 1707-8; Frier, B. M. (2014) Nature Reviews Endocrinology 10(12): 711). One problem is that commercially available insulin analogs are unable to modulate bioactivity in response to circulating glycemia and, therefore, have a narrow therapeutic index. To address this challenge, the concept of a glucose-responsive insulin (GRI), or “smart” insulin, was proposed to mimic the glucose-stimulated insulin secretion in pancreatic beta cells (Brownlee and Cerami (1979) Science 206(4423): 1190-1191; Zaykov et al. (2016) Nature Reviews Drug Discovery 15(6): 425; Bakh et al. (2017) Nature Chemistry 9(10): 937). To date, a number of studies of creating GRI were developed using glucose-triggering signals from lectins (Kaarsholm et al. (2018) Diabetes 67(2): 299-308; Yang et al. (2018) JCI Insight 3(1)), glucose oxidases (Yu et al. (2015) Proceedings of the National Academy of Sciences of the United States of America 112(27): 8260-5; Gu et al. (2013) ACS Nano 7(5): 4194-201), glucose transporters (Wang et al. (2017) Advanced Materials 29(18)), and phenylboronic acid (PBA) (Guo et al. (2015) Advanced Healthcare Materials 4(12): 1796-1801; Chou et al. (2015) Proceedings of the National Academy of Sciences of the United States of America 112(8): 2401-6). The use of PBA to create glucose responsive properties is particularly useful since PBA is smaller in size compared to other sensing agents and is known to bind reversibly to cis-1,2- or cis-1,3-diols such as glucose, thus creating a negative charge on the boronic acid—a property than can be exploited to alter insulin absorption characteristics. Chemically-modified insulin derivatives are therefore promising candidates for GRI designs (Rege et al. (2017) Current Opinion in Endocrinology, Diabetes and Obesity 24(4): 267-278).

Insulin glargine (Lantus®) is a commonly used long-acting insulin for people with diabetes. The protracted mechanism of action for insulin glargine is due to the addition of two arginine residues in the B chain, which increases the isoelectric point (pI) of insulin to 6.7, thus lowering its solubility at physiological pH (Owens and Griffiths (2002) International Journal of Clinical Practice 56(6): 460-466; Heinemann et al. (2000) Diabetes Care 23(5): 644-649). Once injected, insulin glargine precipitates in the injection site and is very slowly converted into hexamers, dimers, and monomers for absorption, thus providing a long-lasting and steady insulin entry into the bloodstream in vivo. Despite the long-lasting benefits of insulin glargine, the addition of glucose-responsive properties to enhance glycemic control, yet prevent iatrogenic hypoglycemia, have remained elusive. These needs and others are met by the present invention.

### BRIEF SUMMARY

In accordance with the purpose(s) of the invention, as embodied and broadly described herein, the invention, in one aspect, relates to insulin-based peptides useful in the treatment of diabetes.

Thus, disclosed are peptides comprising an insulin A chain peptide and an insulin B chain peptide, wherein the insulin B chain peptide comprises at least 32 amino acid residues, and wherein at least three of the amino acid residues of the insulin B chain peptide are lysine residues.

Disclosed are peptides comprising an insulin A chain peptide and an insulin B chain peptide, wherein the B chain peptide comprises a substitution at amino acid 10 and amino acid 20, further comprising at least one substitution in the A chain peptide. In some instances, the at least one substitution in the A chain peptide is T8H, T8Y, T8K, or S9R.

Also disclosed are peptides comprising an insulin A chain peptide and an insulin B chain peptide, wherein the peptide is directly conjugated to at least one organic borate group.

Also disclosed are methods of making a disclosed peptide.

Also disclosed are methods of making an insulin B chain peptide, wherein the insulin B chain peptide is directly conjugated to an organic borate group, the method comprising the step of reacting a peptide-bound insulin B chain resin with a phenylboronic acid having a structure represented by a formula:

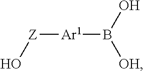

wherein Z is selected from C(O) and SO₂; wherein Ar¹ is selected from 5-membered aryl, 5-membered heteroaryl, 6-membered aryl, and 6-membered heteroaryl and is substituted with 0, 1, 2, or 3 groups independently selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino, and cleaving the resin, thereby making the insulin B chain peptide.

Also disclosed are pharmaceutical compositions comprising a therapeutically effective amount of a disclosed peptide and a pharmaceutically acceptable carrier.

Also disclosed are methods of treating diabetes in a subject, the method comprising administering to the subject a therapeutically effective amount of a disclosed peptide, thereby treating diabetes in the subject.

Also disclosed are methods of modifying insulin receptor activation in at least one cell, the method comprising contacting at least one cell with an effective amount of a disclosed peptide, thereby increasing insulin receptor activation in at least one cell.

Also disclosed are methods of lowering blood sugar in a subject, the method comprising administering to the subject a therapeutically effective amount of a disclosed peptide, thereby lowering blood sugar in the subject.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1A-C show representative schematics of liquid chromatography (LC) traces (top panel) and mass spectrometry (MS) spectra of compound no. 5 (InsA(G), FIG. 1A), compound no. 9 InsB, (FIG. 1B), and compound no. 10 (smart glargine, FIG. 1C). Compound nos. correspond to those shown in FIG. 4B.

FIG. 2 shows a representative image of an insulin analogs having a phenylboronic acid-containing residue.

FIG. 3 shows a representative schematic illustrating the chemical synthesis of smart glargine.

FIG. 4A and FIG. 4B show representative schematics illustrating a proposed design of glucose-responsive smart glargine.

FIG. 5A-E show representative data pertaining to the characterizations of insulin derivatives.

FIG. 6A-D show representative data pertaining to glucose clamp studies of insulin derivatives.

FIG. 7A and FIG. 7B show representative data illustrating the results of an insulin tolerance test.

FIG. 8 shows a representative image illustrating the design and synthesis of glucose responsive insulin derivatives with improved glucose responsiveness.

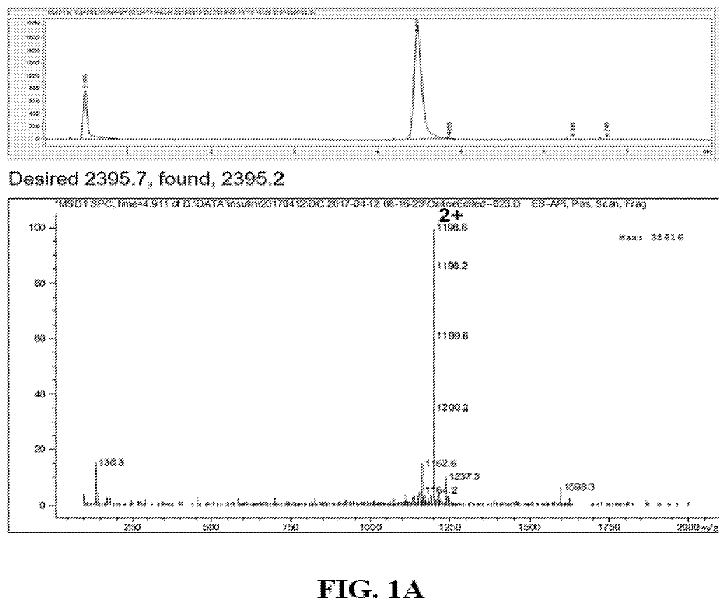

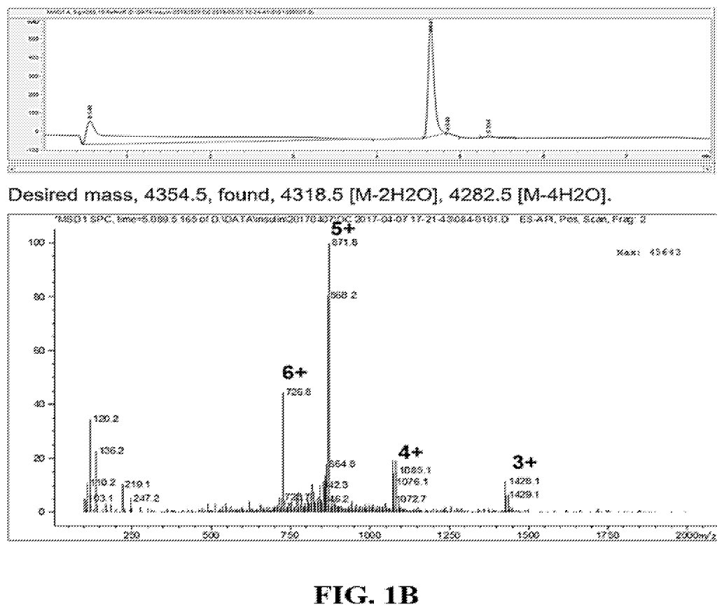

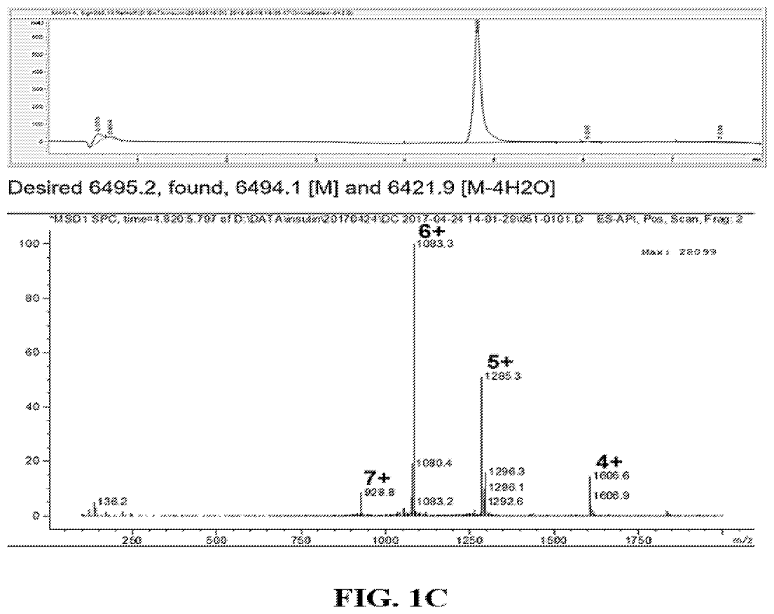

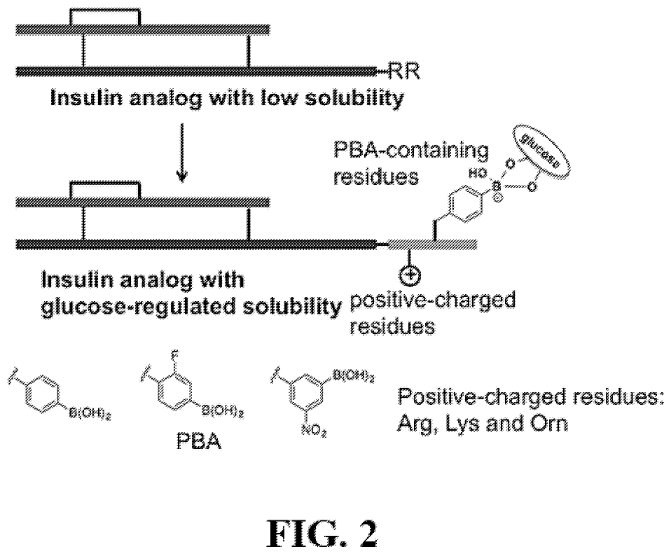

Additional advantages of the invention will be set forth in part in the description that follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the peptide are discussed, each and every combination and permutation of peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, reference to “a peptide” includes a plurality of such peptides, reference to “the peptide” is a reference to one or more peptides and equivalents thereof known to those skilled in the art, and so forth.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range¬from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

The terms “A chain peptide” and “B chain peptide” are interchangeable with “insulin A chain peptide” and “insulin B chain peptide.”

The term “therapeutic” refers to a treatment, therapy, or drug that can treat a disease or condition or that can ameliorate one or more symptoms associated with a disease or condition. As used herein, a therapeutic can refer to a therapeutic compound, including, but not limited to proteins, peptides, nucleic acids (e.g., CpG oligonucleotides), small molecules, vaccines, allergenic extracts, antibodies, gene therapies, other biologics or small molecules.

As used herein, the term “subject” or “patient” refers to any organism to which a peptide or composition of this invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates; and the like.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be type 1 diabetes or any other insulin-related condition.

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. In some aspects of the disclosed methods, the subject has been diagnosed with a need for treatment of disease or disorder such as, for example, diabetes, prior to the administering step. As used herein, the phrase “identified to be in need of treatment for a disorder,” or the like, refers to selection of a subject based upon need for treatment of the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “contacting” as used herein refers to bringing a disclosed compound and a cell, target receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

The term amino acid “modification” or “modified” amino acid refers to a substitution of an amino acid, or the derivation of an amino acid by the addition and/or removal of chemical groups to/from the amino acid, and includes substitution with any of the 20 amino acids commonly found in human proteins, as well as atypical or non-naturally occurring amino acids. Commercial sources of atypical amino acids include Sigma-Aldrich (Milwaukee, Wis.), ChemPep Inc. (Miami, Fla.), and Genzyme Pharmaceuticals (Cambridge, Mass.). Atypical amino acids can be be purchased from commercial suppliers, synthesized de novo, or chemically modified or derivatized from naturally occurring amino acids.

As used herein an amino acid “substitution” refers to the replacement of one amino acid residue by a different amino acid residue. The substituted amino acid may be any of the 20 amino acids commonly found in human proteins, as well as atypical or non-naturally occurring amino acids.

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radio-actively labeled forms, isomers, solvates and combinations thereof. The “combinations” mentioned in this context are refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

“Pharmaceutically acceptable salts” refer to derivatives of the disclosed compounds wherein the parent compound is modified by making acid or base salts thereof. The compounds of this disclosure form acid addition salts with a wide variety of organic and inorganic acids and include the physiologically acceptable salts which are often used in pharmaceutical chemistry. Such salts are also part of this disclosure. Typical inorganic acids used to form such salts include hydrochloric, hydrobromic, hydroiodic, nitric, sulfuric, phosphoric, hypophosphoric acid, and the like. Salts derived from organic acids, such as aliphatic mono- and dicarboxylic acids, phenyl substituted alkanoic acids, hydroxyalkanoic and hydroxyalkandioic acids, aromatic acids, aliphatic and aromatic sulfonic acids may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, β-hydroxybutyrate, butyne-1,4-dioate, hexyne-1,4-dioate, caprate, caprylate, chloride, cinnamate, citrate, formate, fumarate, glycollate, heptanoate, hippurate, lactate, malate, maleate, hydroxymaleate, malonate, mandelate, mesylate, nicotinate, isonicotinate, nitrate, oxalate, phthalate, teraphthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzene-sulfonate, p-bromobenzenesulfonate, chlorobenzenesulfonate, ethanesulfonate, 2-hydroxyethanesulfonate, methanesulfonate, naphthalene-1-sulfonate, naphthalene-2-sulfonate, p-toleunesulfonate, xylenesulfonate, tartarate, and the like.

It is understood that the compounds of the present disclosure relate to all optical isomers and stereo-isomers at the various possible atoms of the molecule, unless specified otherwise. Compounds may be separated or prepared as their pure enantiomers or diasteriomers by crystallization, chromatography or synthesis.

The term “leaving group” refers to an atom (or a group of atoms) with electron withdrawing ability that can be displaced as a stable species, taking with it the bonding electrons. Examples of suitable leaving groups include sulfonate esters, including triflate, mesylate, tosylate, brosylate, and halides.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, optionally substituted alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, optionally substituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be an optionally substituted alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ—or -(A¹O(O)C-A²-OC(O))ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an optionally substituted alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an optionally substituted alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “heterocycle,” as used herein refers to single and multi-cyclic aromatic or non-aromatic ring systems in which at least one of the ring members is other than carbon. Heterocycle includes pyridinde, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridine, pyridazine, pyrimidine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like.

The term “hydroxyl” as used herein is represented by the formula —OH.

The term “ketone” as used herein is represented by the formula A¹C(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “azide” as used herein is represented by the formula —N₃.

The term “nitro” as used herein is represented by the formula —NO₂.

The term “nitrile” as used herein is represented by the formula —CN.

The term “silyl” as used herein is represented by the formula —SiA¹A²A³, where A¹, A², and A³ can be, independently, hydrogen or an optionally substituted alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A′, —S(O)₂A¹, —OS(O)₂A¹, or —OS(O)₂OA¹, where A¹ can be hydrogen or an optionally substituted alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an optionally substituted alkyl, cycloalkyl, alkenyl, cycloalkenyl, alkynyl, cycloalkynyl, aryl, or heteroaryl group as described herein. The term “sulfone” as used herein is represented by the formula A'S(O)₂A², where A¹ and A² can be, independently, an optionally substituted alkyl, cycloalkyl, alkenyl, cycloalkenyl, alkynyl, cycloalkynyl, aryl, or heteroaryl group as described herein. The term “sulfoxide” as used herein is represented by the formula A'S(O)A², where A¹ and A² can be, independently, an optionally substituted alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})²; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}2; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR¹), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C1-6 aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(RT)S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR¹, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

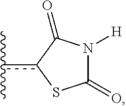

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. “Solvates” refers to the compound formed by the interaction of a solvent and a solute and includes hydrates. Solvates are usually crystalline solid adducts containing solvent molecules within the crystal structure, in either stoichiometric or nonstoichiometric proportions. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvate or water molecules can combine with the compounds according to the invention to form solvates and hydrates. Unless stated to the contrary, the invention includes all such possible solvates.

The term “co-crystal” means a physical association of two or more molecules, which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is known that chemical substances form solids, which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

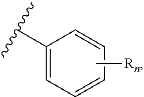

which is understood to be equivalent to a formula:

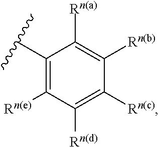

wherein n is typically an integer. That is, Rⁿ is understood to represent five independent substituents, R^{n(a)}, R^{n(b)}, R^{n(c)}, R^{n(d)}, R^{n(e)}. In each such case, each of the five Rⁿ can be hydrogen or a recited substituent. By “independent substituents,” it is meant that each R substituent can be independently defined. For example, if in one instance R^{n(a)} is halogen, then R^{n(b)} is not necessarily halogen in that instance.

In some yet further aspects, a structure of a compound can be represented by a formula:

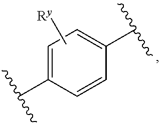

wherein R^{y} represents, for example, 0-2 independent substituents selected from A¹, A², and A³, which is understood to be equivalent to the groups of formulae:
wherein R^{y} represents 0 independent substituents

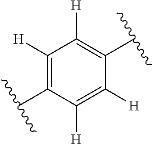

wherein R^{y} represents 1 independent substituent

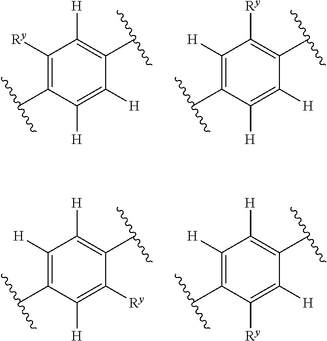

wherein R^{y} represents 2 independent substituents

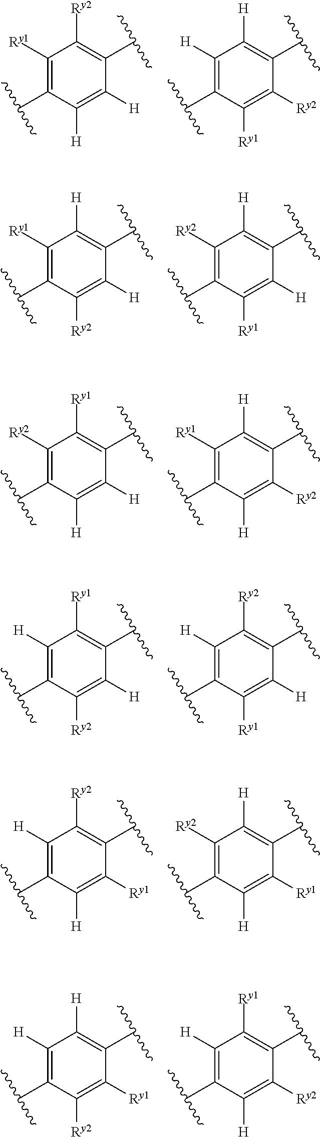

Again, by “independent substituents,” it is meant that each R substituent can be independently defined. For example, if in one instance R^{y1} is A¹, then R^{y2} is not necessarily A¹ in that instance.

In some further aspects, a structure of a compound can be represented by a formula,

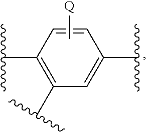

wherein, for example, Q comprises three substituents independently selected from hydrogen and A, which is understood to be equivalent to a formula:

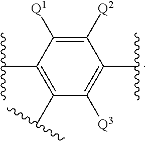

Again, by “independent substituents,” it is meant that each Q substituent is independently defined as hydrogen or A, which is understood to be equivalent to the groups of formulae:
wherein Q comprises three substituents independently selected from H and A

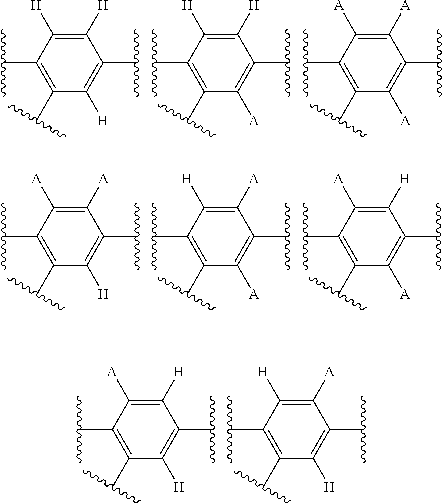

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the materials for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

### B. Peptides

In one aspect, disclosed are peptides comprising an insulin A chain peptide and an insulin B chain peptide, wherein the insulin B chain peptide comprises at least 32 amino acid residues, and wherein at least three of the amino acid residues of the insulin B chain peptide are lysine residues.

In one aspect, disclosed are peptides comprising an insulin A chain peptide and an insulin B chain peptide, wherein the peptide is directly conjugated to at least one organic borate group.

Wild type insulin comprises an A chain peptide and a B chain peptide. Wild type human insulin A chain is represented by the sequence GIVEQCCTSICSLYQLENYCN (SEQ ID NO:1). Wild type human insulin B chain is represented by the sequence FVNQHLCGSHLVEALYLVCGERGFFYTPKT (SEQ ID NO:2).

In various aspects, the insulin A chain peptide and the insulin B chain peptide are bonded via at least one disulfide bond. In a further aspect, the insulin A chain peptide and the insulin B chain peptide are bonded via at least two disulfide bonds.

In various aspects, the disclosed peptides are monomers. In other words, in various aspects, the disclosed peptides are less likely to form dimers, tetramers, hexamers, etc. than wild type insulin.

In various aspects, the insulin A chain peptide is at least 70% identical to wild type human insulin A chain peptide. In some instances, the insulin A chain peptide is at least 60, 65, 70, 75, 80, 85, 90, 95, 99% identical to wild type human insulin A chain peptide. In some instances, the percent identity can be reached by the deletion of one or more amino acids from the N-terminus or C-terminus end of the disclosed peptides.

In various aspects, the insulin A chain peptide comprises the sequence of GIVEQCCTSICSLYQLENYCN (SEQ ID NO:1). In a further aspect, the insulin A chain peptide comprises the sequence of GIVEQCCTSICSLYQLENYCG (SEQ ID NO:3).

In various aspects, the insulin B chain peptide is at least 70% identical to wild type human insulin B chain peptide. In a further aspect, the insulin B chain peptide is at least 60, 65, 70, 75, 80, 85, 90, 95, or 99% identical to wild type human insulin B chain peptide. In a still further aspect, the percent identity can be reached by the deletion of one or more amino acids from the N-terminus or C-terminus end of the disclosed peptides. In yet a further aspect, the percent identity can be reached by the addition of one or more amino acids from the N-terminus or C-terminus end of the disclosed peptides.

In various aspects, the insulin B chain peptide comprises at least 33 amino acid residues. In a further aspect, the insulin B chain peptide comprises at least 34 amino acid residues.

In various aspects, an amino acid at position B29 is a lysine residue. In a further aspect, the B29 lysine residue is modified. In a still further aspect, the B29 lysine residue is not modified.

In various aspects, an amino acid at position B33 is a lysine residue. In a further aspect, the B33 lysine residue is modified. In a still further aspect, the B33 lysine residue is not modified.

In various aspects, an amino acid at position B34 is a lysine residue. In a further aspect, the B34 lysine residue is modified. In a still further aspect, the B34 lysine residue is not modified.

In various aspects, an amino acid at position B29 and an amino acid at position B33 are lysine residues. In a further aspect, an amino acid at position B29 and an amino acid at position B34 are lysine residues. In a still further aspect, an amino acid at position B33 and an amino acid at position B34 are lysine residues. In yet a further aspect, an amino acid at position B29, an amino acid at position B33, and an amino acid at position B34 are lysine residues.

In various aspects, the B29 lysine residue is not modified and each of the B33 and B34 lysine residues are modified. In a further aspect, the B33 lysine residue is not modified and each of the B29 and B34 lysine residues are modified. In a still further aspect, the B34 lysine residue is not modified and each of the B29 and B33 lysine residues are modified. In yet a further aspect, each of the B29, B33, and B34 lysine residues are modified. In an even further aspect, each of the B29, B33, and B34 lysine residues are not modified.

In various aspects, the insulin B chain peptide comprises the sequence of FVNQHLCGSHLVEALYLVCGERGFFYTPKT (SEQ ID NO:2). In a further aspect, the insulin B chain peptide comprises the sequence of FVNQHLCGSHLVEALYLVCGERGFFYTPKTR (SEQ ID NO:4), FVNQHLCGSHLVEALYLVCGERGFFYTPKTRR (SEQ ID NO:5), or FVNQHLCGSHLVEALYLVCGERGFFYTPKTRRR (SEQ ID NO:6). In a still further aspect, the insulin B chain peptide comprises the sequence of FVNQHLCGSHLVEALYLVCGERGFFYTPKTR (SEQ ID NO:4) or FVNQHLCGSHLVEALYLVCGERGFFYTPKTRRR (SEQ ID NO:6). In yet a further aspect, the insulin B chain peptide comprises the sequence of FVNQHLCGSHLVEALYLVCGERGFFYTPKTRR (SEQ ID NO:5) or FVNQHLCGSHLVEALYLVCGERGFFYTPKTRRR (SEQ ID NO:6). In an even further aspect, the insulin B chain peptide comprises the sequence of FVNQHLCGSHLVEALYLVCGERGFFYTPKTR (SEQ ID NO:4). In a still further aspect, the insulin B chain peptide comprises the sequence of FVNQHLCGSHLVEALYLVCGERGFFYTPKTRR (SEQ ID NO:5). In yet a further aspect, the insulin B chain peptide comprises the sequence of FVNQHLCGSHLVEALYLVCGERGFFYTPKTRRR (SEQ ID NO:6).

In various aspects, the insulin B chain peptide comprises the sequence of FVNQHLCGSHLVEALYLVCGERGFFYTPKTRK (SEQ ID NO:7). In a further aspect, the insulin B chain peptide comprises the sequence of FVNQHLCGSHLVEALYLVCGERGFFYTPKTRKK (SEQ ID NO:8).

In various aspects, at least one of the lysine residues is on the insulin B chain peptide's C-terminus. In a further aspect, at least two of the lysine residues are on the insulin B chain peptide's C-terminus. In a still further aspect, three of the lysine residues are on the insulin B chain peptide's C-terminus.

In various aspects, the insulin A chain peptide and the insulin B chain peptide are bonded via at least one disulfide bond, the insulin A chain peptide comprises the sequence of GIVEQCCHRICSLYQLENYCN (SEQ ID NO:1), and the insulin B chain peptide comprises the sequence of FVNQHLCGSHLVEALYLVCGERGFFYTPKTRKK (SEQ ID NO:8). In a further aspect, one or both of the B33 lysine residue and the B34 lysine residue are modified. In a still further aspect, one of the B33 lysine residue and the B34 lysine residue are modified. In yet a further aspect, the B33 lysine residue is modified. In an even further aspect, the B34 lysine residue is modified. In a still further aspect, both of the B33 lysine residue and the B34 lysine residue are modified.

In various aspects, the insulin A chain peptide comprises the sequence of GIVEQCCTSICSLYQLENYCN (SEQ ID NO:1) or GIVEQCCTSICSLYQLENYCGSEQ ID NO:3) and the insulin B chain peptide comprises the sequence of FVNQHLCGSHLVEALYLVCGERGFFYTPKT (SEQ ID NO:2), FVNQHLCGSHLVEALYLVCGERGFFYTPKTRR (SEQ ID NO:5), or FVNQHLCGSHLVEALYLVCGERGFFYTPKTRKK (SEQ ID NO:8). In a further aspect, the insulin A chain peptide comprises the sequence of GIVEQCCTSICSLYQLENYCN (SEQ ID NO:1) or GIVEQCCTSICSLYQLENYCGSEQ ID NO:3) and the insulin B chain peptide comprises the sequence of FVNQHLCGSHLVEALYLVCGERGFFYTPKTRR (SEQ ID NO:5) or FVNQHLCGSHLVEALYLVCGERGFFYTPKTRKK (SEQ ID NO:8). In a still further aspect, the insulin A chain peptide comprises the sequence of GIVEQCCTSICSLYQLENYCN (SEQ ID NO:1) or GIVEQCCTSICSLYQLENYCG (SEQ ID NO:3) and the insulin B chain peptide comprises the sequence of FVNQHLCGSHLVEALYLVCGERGFFYTPKTRKK (SEQ ID NO:8).

In various aspects, the insulin A chain peptide comprises the sequence of GIVEQCCTSICSLYQLENYCN (SEQ ID NO:1) and the insulin B chain peptide comprises the sequence of FVNQHLCGSHLVEALYLVCGERGFFYTPKT (SEQ ID NO:2), FVNQHLCGSHLVEALYLVCGERGFFYTPKTRR (SEQ ID NO:5), or FVNQHLCGSHLVEALYLVCGERGFFYTPKTRKK (SEQ ID NO:8). In a further aspect, the insulin A chain peptide comprises the sequence of GIVEQCCTSICSLYQLENYCG (SEQ ID NO:3) and the insulin B chain peptide comprises the sequence of FVNQHLCGSHLVEALYLVCGERGFFYTPKT (SEQ ID NO:2), FVNQHLCGSHLVEALYLVCGERGFFYTPKTRR (SEQ ID NO:5), or FVNQHLCGSHLVEALYLVCGERGFFYTPKTRKK (SEQ ID NO:8).

In various aspects, the insulin A chain peptide comprises the sequence of GIVEQCCTSICSLYQLENYCG (SEQ ID NO:3), the insulin B chain peptide comprises the sequence of FVNQHLCGSHLVEALYLVCGERGFFYTPKTRKK (SEQ ID NO:8), the B29 lysine residue is not modified, each of the B33 lysine residue and the B34 lysine residue is directly conjugated to an organic borate group, and each occurrence of the organic borate group has a structure represented by a formula:

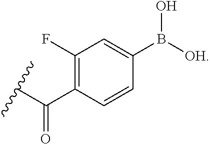 [SMILES: *C(=O)c1ccc(B(O)O)cc1F]

In various aspects, the peptide is directly conjugated to two organic borate groups.

In various aspects, the peptide is directly conjugated to at least one organic borate group via a lysine residue. In a further aspect, the peptide is directly conjugated to one organic borate group via a lysine residue. In a still further aspect, the peptide is directly conjugated to two organic borate groups via two lysine residues.

In various aspects, the disclosed peptides can comprise one or more unnatural amino acids, modified amino acids, or synthetic amino acid analogues. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, cyclopentylalanine, β-alanine, fluoro-amino acids, designer amino acids such as β-methyl amino acids, Cα-methyl amino acids, Nα-methyl amino acids, and amino acid analogues in general. Also included within the scope are peptides which are differentially modified during or after synthesis by, for example, biotinylation, benzylation, glycosylation, acetylation, phosphorylation, amidation, derivatization by known protecting/blocking groups, proteolytic cleavage, linkage to an antibody molecule or other cellular ligand, etc. Without wishing to be bound by theory, these modifications may serve to increase the stability and/or bioactivity of the peptide.

In various aspects, disclosed are therapeutic proteins having an A chain peptide bonded to a B chain peptide via at least one disulfide bond, wherein the insulin B chain peptide comprises at least 32 amino acid residues, and wherein at least three of the amino acid residues of the insulin B chain peptide are lysine residues. Without wishing to be bound by theory, it is appreciated that the disclosed therapeutic proteins can be employed in pharmaceutical compositions and used in connection with treatment of disorders such as, for example, diabetes.

1. Organic Borate Groups

In one aspect, the disclosed peptides are directly conjugated to at least one organic borate group. In a further aspect, the disclosed peptides are directly conjugated to one organic borate group. In a still further aspect, the disclosed peptides are directly conjugated to a plurality of organic borate groups. In yet a further aspect, the disclosed peptides are directly conjugated to two organic borate groups.

In various aspects, the organic borate group has a structure represented by a formula:

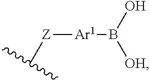

wherein Z is selected from C(O) and SO₂; wherein Ar¹ is selected from 5-membered aryl, 5-membered heteroaryl, 6-membered aryl, and 6-membered heteroaryl and is substituted with 0, 1, 2, or 3 groups independently selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino.

In various aspects, the organic borate group has a structure represented by a formula:

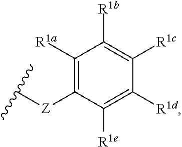

wherein each of R^{1a}, R^{1b}, R^{1c}, R^{1d}, and R^{1e} is independently selected from hydrogen, halogen, —CN, —NO₂, —OH, C1-C4 alkyl, C1-C4 haloalkyl, C1-C4 hydroxyalkyl, C1-C4 alkoxy, C1-C4 alkylamino, (C1-C4)(C1-C4) dialkylamino, and —B(OH)₂, provided that one and only one of R^{1a}, R^{1b}, R^{1c}, R^{1d}, and R^{1e} is —B(OH)₂.

In various aspects, the organic borate group has a structure represented by a formula:

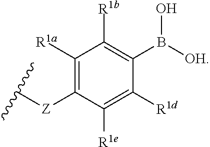

In various aspects, the organic borate group has a structure represented by a formula:

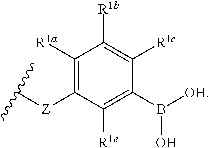

In various aspects, the organic borate group has a structure represented by a formula:

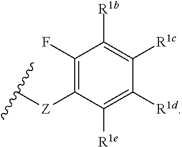

In various aspects, the organic borate group has a structure represented by a formula:

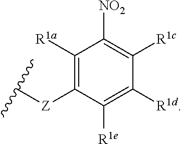

In various aspects, the organic borate group has a structure represented by a formula:

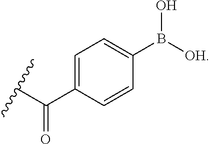 [SMILES: *C(=O)c1ccc(B(O)O)cc1]

In various aspects, the organic borate group has a structure represented by a formula:

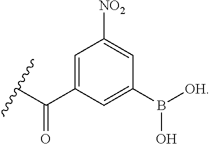 [SMILES: *C(=O)c1cc(B(O)O)cc([N+](=O)[O-])c1]

In various aspects, the organic borate group has a structure represented by a formula:

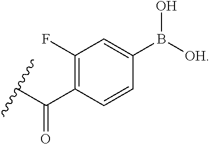 [SMILES: *C(=O)c1ccc(B(O)O)cc1F]

a. Z Groups

In one aspect, Z is selected from C(O) and SO₂. In a further aspect, Z is C(O). In a still further aspect, Z is SO₂.

b. R^{1A}, R^{1B}, R^{1c}, R^{1D}, AND R^{1E} Groups

In one aspect, each of R^{1a}, R^{1b}, R^{1c}, R^{1d}, and R^{1e} is independently selected from hydrogen, halogen, —CN, —NO₂, —OH, C1-C4 alkyl, C1-C4 haloalkyl, C1-C4 hydroxyalkyl, C1-C4 alkoxy, C1-C4 alkylamino, (C1-C4)(C1-C4) dialkylamino, and —B(OH)₂, provided that one and only one of R^{1a}, R^{1b}, R^{1c}, R^{1d}, and R^{1e} is —B(OH)₂. In a further aspect, each of R^{1a}, R^{1b}, R^{1c}, R^{1d}, and R^{1e} is independently selected from hydrogen and —B(OH)₂.

In a further aspect, each of R^{1a}, R^{1b}, R^{1c}, R^{1d}, and R^{1e} is independently selected from hydrogen, —F, —Cl, —CN, —NO₂, —OH, C1-C4 alkyl, C1-C4 haloalkyl, C1-C4 hydroxyalkyl, C1-C4 alkoxy, C1-C4 alkylamino, (C1-C4)(C1-C4) dialkylamino, and —B(OH)₂. In a still further aspect, each of R^{1a}, R^{1b}, R^{1c}, R^{1d}, and R^{1e} is independently selected from hydrogen, —F, —Cl, —CN, —NO₂, —OH, methyl, ethyl, n-propyl, isopropyl, —CF₃, —CHF₂, —CH₂F, —CH₂CH₂F, —CH(CH₃)CH₂F, —CH₂CH₂CH₂F, —CCl₃, —CHCl₂, —CH₂Cl, —CH₂CH₂Cl, —CH(CH₃)CH₂Cl, —CH₂CH₂CH₂Cl, —CH₂OH, —CH₂CH₂OH, —CH(CH₃)CH₂OH, —CH₂CH₂CH₂OH, —OCH₃, —OCH₂CH₃, —OCH(CH₃)₂, —OCH₂CH₂CH₃, —NHCH₃, —NHCH₂CH₃, —NHCH(CH₃)₂, —NHCH₂CH₂CH₃, —N(CH₃)₂, —N(CH₃)CH₂CH₃, —N(CH₂CH₃)CH₂CH₃, —N(CH₃)CH(CH₃)₂, —N(CH₃)CH₂CH₂CH₃, and —B(OH)₂. In yet a further aspect, each of R^{1a}, R^{1b}, R^{1c}, R^{1d}, and R^{1e} is independently selected from hydrogen, —F, —Cl, —CN, —NO₂, —OH, methyl, ethyl, —CF₃, —CHF₂, —CH₂F, —CH₂CH₂F, —CCl₃, —CHCl₂, —CH₂Cl, —CH₂CH₂Cl, —CH₂OH, —CH₂CH₂OH, —OCH₃, —OCH₂CH₃, —NHCH₃, —NHCH₂CH₃, —N(CH₃)₂, —N(CH₃)CH₂CH₃, —N(CH₂CH₃)CH₂CH₃, and —B(OH)₂. In an even further aspect, each of R^{1a}, R^{1b}, R^{1c}, R^{1d}, and R^{1e} is independently selected from hydrogen, —F, —Cl, —CN, —NO₂, —OH, methyl, —CF₃, —CHF₂, —CH₂F, —CCl₃, —CHCl₂, —CH₂Cl, —CH₂OH, —OCH₃, —OCH₂CH₃, —NHCH₃, —N(CH₃)₂, and —B(OH)₂.

In a further aspect, each of R^{1a}, R^{1b}, R^{1c}, R^{1d}, and R^{1e} is independently selected from hydrogen, halogen, and —B(OH)₂. In a still further aspect, each of R^{1a}, R^{1b}, R^{1c}, R^{id}, and R^{1e} is independently selected from hydrogen, —F, —Cl, —Br, and —B(OH)₂. In yet a further aspect, each of R^{1a}, R^{1b}, R^{1c}, R^{1d}, and R^{1e} is independently selected from hydrogen, —F, —Cl and —B(OH)₂. In an even further aspect, each of R^{1a}, R^{1b}, R^{1c}, R^{1d}, and R^{1e} is independently selected from hydrogen, —F, and —B(OH)₂.

In a further aspect, R^{1a} is —B(OH)₂. In a still further aspect, R^{1b} is —B(OH)₂. In yet a further aspect, R^{1c} is —B(OH)₂. In an even further aspect, R^{1d} is —B(OH)₂. In a still further aspect, R^{1e} is —B(OH)₂.

In a further aspect, one of R^{1a}, R^{1b}, R^{1c}, R^{1d}, and R^{1e} is halogen. In a still further aspect, one of R^{1a}, R^{1b}, R^{1c}, R^{id}, and R^{1e} is —F.

In a further aspect, R^{1a} is halogen. In a still further aspect, R^{1a} is —F.

c. AR¹ Groups

In one aspect, Ar¹ is selected from 5-membered aryl, 5-membered heteroaryl, 6-membered aryl, and 6-membered heteroaryl and is substituted with 0, 1, 2, or 3 groups independently selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In a further aspect, Ar¹ is selected from 5-membered aryl, 5-membered heteroaryl, 6-membered aryl, and 6-membered heteroaryl and is substituted with 0, 1, or 2 groups independently selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In a still further aspect, Ar¹ is selected from 5-membered aryl, 5-membered heteroaryl, 6-membered aryl, and 6-membered heteroaryl and is substituted with 0 or 1 group selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In yet a further aspect, Ar¹ is selected from 5-membered aryl, 5-membered heteroaryl, 6-membered aryl, and 6-membered heteroaryl and is monosubstituted with a group selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In an even further aspect, Ar¹ is selected from 5-membered aryl, 5-membered heteroaryl, 6-membered aryl, and 6-membered heteroaryl and is unsubstituted.

In various aspects, Ar¹ is selected from 5-membered aryl and 5-membered heteroaryl and is substituted with 0, 1, 2, or 3 groups independently selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In a further aspect, Ar¹ is selected from 5-membered aryl and 5-membered heteroaryl and is substituted with 0, 1, or 2 groups independently selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In a still further aspect, Ar¹ is selected from 5-membered aryl and 5-membered heteroaryl and is substituted with 0 or 1 group selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In yet a further aspect, Ar¹ is selected from 5-membered aryl and 5-membered heteroaryl and is monosubstituted with a group selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In an even further aspect, Ar¹ is selected from 5-membered aryl and 5-membered heteroaryl and is unsubstituted.

In various aspects, Ar¹ is 5-membered aryl substituted with 0, 1, 2, or 3 groups independently selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In a further aspect, Ar¹ is 5-membered aryl substituted with 0, 1, or 2 groups independently selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In a still further aspect, Ar¹ is 5-membered aryl is substituted with 0 or 1 group selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In yet a further aspect, Ar¹ is 5-membered aryl monosubstituted with a group selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In an even further aspect, Ar¹ is unsubstituted 5-membered aryl.

In various aspects, Ar¹ is 5-membered heteroaryl substituted with 0, 1, 2, or 3 groups independently selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. Examples of 5-membered heteroaryls include, but are not limited to, furanyl, pyrrolyl, thiophenyl, imidazolyl, pyraolyl, oxazolyl, isoxazolyl, and thiazolyl. In a further aspect, Ar¹ is 5-membered heteroaryl substituted with 0, 1, or 2 groups independently selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In a still further aspect, Ar¹ is 5-membered heteroaryl is substituted with 0 or 1 group selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In yet a further aspect, Ar¹ is 5-membered heteroaryl monosubstituted with a group selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In an even further aspect, Ar¹ is unsubstituted 5-membered heteroaryl.

In various aspects, Ar¹ is selected from 6-membered aryl and 6-membered heteroaryl and is substituted with 0, 1, 2, or 3 groups independently selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In a further aspect, Ar¹ is selected from 6-membered aryl and 6-membered heteroaryl and is substituted with 0, 1, or 2 groups independently selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In a still further aspect, Ar¹ is selected from 6-membered aryl and 6-membered heteroaryl and is substituted with 0 or 1 group selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In yet a further aspect, Ar¹ is selected from 6-membered aryl and 6-membered heteroaryl and is monosubstituted with a group selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In an even further aspect, Ar¹ is selected from 6-membered aryl and 6-membered heteroaryl and is unsubstituted.

In various aspects, Ar¹ is 6-membered aryl substituted with 0, 1, 2, or 3 groups independently selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In a further aspect, Ar¹ is 6-membered aryl substituted with 0, 1, or 2 groups independently selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In a still further aspect, Ar¹ is 6-membered aryl is substituted with 0 or 1 group selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In yet a further aspect, Ar¹ is 6-membered aryl monosubstituted with a group selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In an even further aspect, Ar¹ is unsubstituted 6-membered aryl.

In various aspects, Ar¹ is 6-membered heteroaryl substituted with 0, 1, 2, or 3 groups independently selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. Examples of 6-membered heteroaryls include, but are not limited to, pyridinyl, pyrazinyl, pyrimidinyl, pyridazinyl, 1,2,3-triazinyl, 1,2,4-triazinyl, and 1,3,5-triazinyl. In a further aspect, Ar¹ is 6-membered heteroaryl substituted with 0, 1, or 2 groups independently selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In a still further aspect, Ar¹ is 6-membered heteroaryl is substituted with 0 or 1 group selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In yet a further aspect, Ar¹ is 6-membered heteroaryl monosubstituted with a group selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino. In an even further aspect, Ar¹ is unsubstituted 6-membered heteroaryl.

### C. Pharmaceutical Compositions

In one aspect, disclosed are pharmaceutical compositions comprising a therapeutically effective amount of one or more of the disclosed peptides and a pharmaceutically acceptable carrier. Thus, in various aspects, disclosed are pharmaceutical compositions comprising a therapeutically effective amount of a peptide comprising an insulin A chain peptide and an insulin B chain peptide, wherein the insulin B chain peptide comprises at least 32 amino acid residues, and wherein at least three of the amino acid residues of the insulin B chain peptide are lysine residues. In various further aspects, disclosed are pharmaceutical compositions comprising a therapeutically effective amount of a peptide comprising an insulin A chain peptide and an insulin B chain peptide, wherein the peptide is directly conjugated to at least one organic borate group.

In various aspects, a composition is disclosed comprising an insulin derivative with glucose-dependent solubility. In a further aspect, the isoelectric point (pI) of the insulin derivative composition decreases upon glucose binding due to the generation of the negative charge. Thus, during low blood glucose conditions, the insulin remains micro-crystals like insulin glargine; however, when blood glucose levels are elevated, the solubility increases, which results in the insulin becoming monomeric, increasing bioavailability.

In various aspects, the disclosed peptides can be formulated and/or administered in or with a pharmaceutically acceptable carrier. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Thus, the compositions disclosed herein can comprise lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subjects lung cells. Regarding liposomes, see, e.g., Brigham et al. Am. J. Resp. Cell. Mol. Biol. 1:95 100 (1989); Felgner et al. Proc. Natl. Acad. Sci USA 84:7413 7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In various aspects, disclosed are pharmaceutical compositions comprising any of the disclosed peptides described herein, or a pharmaceutically acceptable salt or solvate thereof, and a pharmaceutically acceptable carrier, buffer, or diluent. In various aspects, the peptide of the pharmaceutical composition is encapsulated in a delivery vehicle. In a further aspect, the delivery vehicle is a liposome, a microcapsule, or a nanoparticle. In a still further aspect, the delivery vehicle is PEG-ylated.

In the methods described herein, delivery of the compositions to cells can be via a variety of mechanisms. As defined above, disclosed herein are compositions comprising any one or more of the peptides described herein and can also include a carrier such as a pharmaceutically acceptable carrier. For example, disclosed are pharmaceutical compositions comprising the peptides disclosed herein, and a pharmaceutically acceptable carrier.

In various aspects, disclosed are pharmaceutical compositions comprising the disclosed peptides. That is, a pharmaceutical composition can be provided comprising a therapeutically effective amount of at least one disclosed peptide or at least one product of a disclosed method and a pharmaceutically acceptable carrier.

In various aspects, the disclosed pharmaceutical compositions comprise the disclosed peptides (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In various aspects, disclosed are pharmaceutical compositions comprising a pharmaceutically acceptable carrier or diluent and, as active ingredient, a therapeutically effective amount of a disclosed peptide, a product of a disclosed method of making, a pharmaceutically acceptable salt, solvate, or polymorph thereof, a hydrate thereof, a solvate thereof, a polymorph thereof, or a stereochemically isomeric form thereof. In a further aspect, a disclosed peptide, a product of a disclosed method of making, a pharmaceutically acceptable salt, solvate, or polymorph thereof, a hydrate thereof, a solvate thereof, a polymorph thereof, or a stereochemically isomeric form thereof, or any subgroup or combination thereof may be formulated into various pharmaceutical forms for administration purposes.

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids,” includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

For therapeutic use, salts of the disclosed compounds are those wherein the counterion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are included within the ambit of the present invention.

The pharmaceutically acceptable acid and base addition salts as mentioned hereinabove or hereinafter are meant to comprise the therapeutically active non-toxic acid and base addition salt forms which the disclosed compounds are able to form. The pharmaceutically acceptable acid addition salts can conveniently be obtained by treating the base form with such appropriate acid. Appropriate acids comprise, for example, inorganic acids such as hydrohalic acids, e.g., hydrochloric or hydrobromic acid, sulfuric, nitric, phosphoric and the like acids; or organic acids such as, for example, acetic, propanoic, hydroxyacetic, lactic, pyruvic, oxalic (i.e., ethanedioic), malonic, succinic (i.e., butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The disclosed compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g., the lithium, sodium, potassium, magnesium, calcium salts and the like, salts with organic bases, e.g., primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form.

In practice, the peptides described herein, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof.

Thus, the pharmaceutical compositions of this invention can include a pharmaceutically acceptable carrier and a compound or a pharmaceutically acceptable salt of the compounds of the invention. By “pharmaceutically acceptable” is meant a material or carrier that would be selected to minimize any degradation of the active ingredient and to minimize any adverse side effects in the subject, as would be well known to one of skill in the art. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. Other examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, Pa. 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

In order to enhance the solubility and/or the stability of the disclosed peptides in pharmaceutical compositions, it can be advantageous to employ α-, β- or γ-cyclodextrins or their derivatives, in particular hydroxyalkyl substituted cyclodextrins, e.g., 2-hydroxypropyl-β-cyclodextrin or sulfobutyl-β-cyclodextrin. Also co-solvents such as alcohols may improve the solubility and/or the stability of the compounds according to the invention in pharmaceutical compositions.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Because of the ease in administration, oral administration is preferred, and tablets and capsules represent the most advantageous oral dosage unit forms in which case solid pharmaceutical carriers are obviously employed. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

A tablet containing the compositions of the present invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a peptide such as sPRR (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. Typically, the final injectable form should be sterile and should be effectively fluid for easy syringability. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations that are intended to be converted, shortly before use, to liquid form preparations.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot on, as an ointment.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Formulations for optical administration may include ointments, lotions, creams, gels, drops, suppositories, sprays, liquids and powders. Conventional pharmaceutical carriers, aqueous, powder or oily bases, thickeners and the like may be desirable.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a disclosed peptide, and/or pharmaceutically acceptable salts thereof, can also be prepared in powder or liquid concentrate form.

The exact dosage and frequency of administration depends on the particular disclosed peptide, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compositions.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In the treatment conditions that require increasing insulin receptor activity an appropriate dosage level will generally be about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900 and 1000 milligrams of the active ingredient for the symptomatic adjustment of the dosage of the patient to be treated. The composition can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific composition employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

In a further aspect, a dosage can be 100U-300U vial, for example, a 100U-200U vial, a 200U-300U vial, or a 150U-250U vial. It can be taken once a day or multiple times a day. In various aspects, it can be taken daily, weekly or monthly.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The present invention is further directed to a method for the manufacture of a medicament for modulating insulin receptor activity (e.g., treatment of type 1 diabetes) in mammals (e.g., humans) comprising combining one or more disclosed peptides or compositions with a pharmaceutically acceptable carrier or diluent. Thus, in one aspect, the invention relates to a method for manufacturing a medicament comprising combining at least one disclosed peptide with a pharmaceutically acceptable carrier or diluent.

The disclosed pharmaceutical compositions can further comprise other therapeutically active compounds, which are usually applied in the treatment of insulin-related conditions.

It is understood that the disclosed compositions can be prepared from the disclosed peptides. It is also understood that the disclosed compositions can be employed in the disclosed methods of using.

As already mentioned, the invention relates to a pharmaceutical composition comprising a therapeutically effective amount of a disclosed peptide, a pharmaceutically acceptable salt, solvate, or polymorph thereof, a hydrate thereof, a solvate thereof, a polymorph thereof, and a pharmaceutically acceptable carrier. Additionally, the invention relates to a process for preparing a pharmaceutical composition, characterized in that a pharmaceutically acceptable carrier is intimately mixed with a therapeutically effective amount of a disclosed peptide.

As already mentioned, the invention also relates to a pharmaceutical composition comprising a disclosed peptide, a pharmaceutically acceptable salt, solvate, or polymorph thereof, and one or more other drugs in the treatment, prevention, control, amelioration, or reduction of risk of diseases or conditions for a disclosed peptide or the other drugs may have utility as well as to the use of such a composition for the manufacture of a medicament. The present invention also relates to a combination of disclosed peptides, a pharmaceutically acceptable salt, solvate, or polymorph thereof, and an anti-cancer therapeutic agent. In various further aspects, the present invention also relates to a combination of disclosed peptides, a pharmaceutically acceptable salt, solvate, or polymorph thereof. The present invention also relates to such a combination for use as a medicine. The different drugs of such a combination or product may be combined in a single preparation together with pharmaceutically acceptable carriers or diluents, or they may each be present in a separate preparation together with pharmaceutically acceptable carriers or diluents.

In various aspects, the disclosed peptides can be administered in an amount of 10-300 μg/kg/day. In a further aspect, the dosing regimen can include a single administration of one or more of the disclosed peptides. In a still further aspect, the dosing regimen can include administering one or more of the disclosed peptides once a week, twice a week, three times a week, four times a week, five times a week, six times a week, or seven times a week for 1, 2, 3, 4, 5, 6, 7, 8, 9, 10, 11, 12, 13, 14, 15, 16, 17, 18, 19, 20, 21, 22, 23, 24, 26, 28, 30, 32, 34, 36, 38, 40, 42, 44, 46, 48, 50 or 52 weeks.

In various aspects, the disclosed peptides can be administered via a needle and syringe, a pen, a pump, an inhaler, an injection port, or a jet injector.

### D. Methods of Making a Peptide

In one aspect, disclosed are methods of making a disclosed peptide. Thus, in various aspects, disclosed are methods of making an insulin B chain peptide, wherein the insulin B chain peptide is directly conjugated to an organic borate group, the method comprising the step of reacting a peptide-bound insulin B chain resin with a phenylboronic acid having a structure represented by a formula:

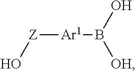

wherein Z is selected from C(O) and SO₂; wherein Ar¹ is selected from 5-membered aryl, 5-membered heteroaryl, 6-membered aryl, and 6-membered heteroaryl and is substituted with 0, 1, 2, or 3 groups independently selected from halogen, —CN, —NO₂, —OH, C1-C4 alkyl, C1-C4 haloalkyl, C1-C4 hydroxyalkyl, C1-C4 alkoxy, C1-C4 alkylamino, and (C1-C4)(C1-C4) dialkylamino, and cleaving the resin, thereby making the insulin B chain peptide.

In various aspects, an amino acid at position B29 is a lysine residue. In a further aspect, the B29 lysine residue is modified. In a still further aspect, the B29 lysine residue is not modified.

In various aspects, an amino acid at position B33 is a lysine residue. In a further aspect, the B33 lysine residue is modified. In a still further aspect, the B33 lysine residue is not modified.

In various aspects, an amino acid at position B34 is a lysine residue. In a further aspect, the B34 lysine residue is modified. In a still further aspect, the B34 lysine residue is not modified.

In various aspects, an amino acid at position B29 and an amino acid at position B33 are lysine residues. In a further aspect, an amino acid at position B29 and an amino acid at position B34 are lysine residues. In a still further aspect, an amino acid at position B33 and an amino acid at position B34 are lysine residues. In yet a further aspect, an amino acid at position B29, an amino acid at position B33, and an amino acid at position B34 are lysine residues.

In various aspects, the B29 lysine residue is not modified and each of the B33 and B34 lysine residues are modified. In a further aspect, the B33 lysine residue is not modified and each of the B29 and B34 lysine residues are modified. In a still further aspect, the B34 lysine residue is not modified and each of the B29 and B33 lysine residues are modified. In yet a further aspect, each of the B29, B33, and B34 lysine residues are modified. In an even further aspect, each of the B29, B33, and B34 lysine residues are not modified.

In various aspects, the insulin B chain peptide comprises the sequence of FVNQHLCGSHLVEALYLVCGERGFFYTPKTRKK (SEQ ID NO:8).

In various aspects, the insulin A chain peptide comprises the sequence of GIVEQCCTSICSLYQLENYCG (SEQ ID NO:3), the insulin B chain peptide comprises the sequence of FVNQHLCGSHLVEALYLVCGERGFFYTPKTRKK (SEQ ID NO:8), the B29 lysine residue is not modified, each of the B33 lysine residue and the B34 lysine residue is directly conjugated to an organic borate group, and each occurrence of the organic borate group has a structure represented by a formula:

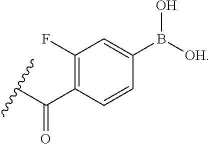 [SMILES: *C(=O)c1ccc(B(O)O)cc1F]

In various aspects, the method further comprises the step of coupling the insulin B chain peptide with an insulin A chain peptide.

### E. Methods of Modifying Insulin Receptor Activation

In one aspect, disclosed are methods of modifying insulin receptor activation in a subject, the method comprising administering an effective amount of any one of the disclosed peptides or pharmaceutical compositions to a subject in need thereof. In a further aspect, a subject in need thereof can be a subject known to have decreased insulin receptor activation compared to a standard activation level. In a still further aspect, a standard activation level of insulin receptor activation can be based on established levels in healthy individuals. In yet a further aspect, a standard activation level of insulin receptor activation can be based on established levels in the subject being treated prior to the determination of a need for increased insulin receptor activation.

In one aspect, disclosed are methods of modifying insulin receptor activation in at least one cell, the method comprising contacting at least one cell with an effective amount of any one of the disclosed peptides or pharmaceutical compositions, thereby increasing insulin receptor activation in at least one cell.

In various aspects, modifying is increasing.

For example, disclosed are methods of modifying insulin receptor activation in a subject, the method comprising administering to the subject an effective amount of a peptide comprising an insulin A chain peptide and an insulin B chain peptide, wherein the insulin B chain peptide comprises at least 32 amino acid residues, and wherein at least three of the amino acid residues of the insulin B chain peptide are lysine residue, thereby modifying insulin receptor activation in the subject. In addition, disclosed are methods of modifying insulin receptor activation in a subject, the method comprising administering to the subject an effective amount of a peptide comprising an insulin A chain peptide and an insulin B chain peptide, wherein the peptide is directly conjugated to at least one organic borate group, thereby modifying insulin receptor activation in the subject.

For example, disclosed are methods of modifying insulin receptor activation in at least one cell, the method comprising contacting at least one cell with an effective amount of a peptide comprising an insulin A chain peptide and an insulin B chain peptide, wherein the insulin B chain peptide comprises at least 32 amino acid residues, and wherein at least three of the amino acid residues of the insulin B chain peptide are lysine residue, thereby modifying insulin receptor activation in at least one cell. In addition, disclosed are methods of modifying insulin receptor activation in at least one cell, the method comprising contacting at least one cell with an effective amount of a peptide comprising an insulin A chain peptide and an insulin B chain peptide, wherein the peptide is directly conjugated to at least one organic borate group, thereby modifying insulin receptor activation in at least one cell.

In various aspects, the insulin A chain peptide and the insulin B chain peptide are bonded via at least one disulfide bond, the insulin A chain peptide comprises the sequence of GIVEQCCHRICSLYQLENYCN (SEQ ID NO:1), and the insulin B chain peptide comprises the sequence of FVNQHLCGSHLVEALYLVCGERGFFYTPKTRKK (SEQ ID NO:8). In a further aspect, one or both of the B33 lysine residue and the B34 lysine residue are modified. In a still further aspect, one of the B33 lysine residue and the B34 lysine residue are modified. In yet a further aspect, the B33 lysine residue is modified. In an even further aspect, the B34 lysine residue is modified. In a still further aspect, both of the B33 lysine residue and the B34 lysine residue are modified.

In various aspects, the insulin A chain peptide comprises the sequence of GIVEQCCTSICSLYQLENYCG (SEQ ID NO:3), the insulin B chain peptide comprises the sequence of FVNQHLCGSHLVEALYLVCGERGFFYTPKTRKK (SEQ ID NO:8), the B29 lysine residue is not modified, each of the B33 lysine residue and the B34 lysine residue is directly conjugated to an organic borate group, and each occurrence of the organic borate group has a structure represented by a formula:

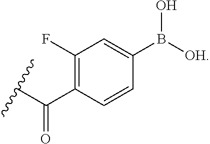 [SMILES: *C(=O)c1ccc(B(O)O)cc1F]

In various aspects, the cell is mammalian. In a further aspect, the cell is human.

In various aspects, contacting is via administration to a subject. In a further aspect, the subject has been diagnosed with a need for treatment of diabetes prior to the administering step. In a still further aspect, the method further comprises the step of identifying a subject in need of treatment of diabetes.

In various aspects, diabetes is type 1 diabetes. In a further aspect, diabetes is type 2 diabetes. In a still further aspect, diabetes is gestational diabetes.

In various aspects, the subject is a mammal. In a further aspect, the mammal is a human.

### F. Methods of Lowering Blood Sugar

In one aspect, disclosed are methods of lowering blood sugar in a subject, the method comprising administering a therapeutically effective amount of any one of the disclosed peptides or pharmaceutical compositions to a subject in need thereof. In various aspects, a subject in need thereof can be a subject known to have increased blood sugar compared to a standard blood sugar level. In a further aspect, a standard activation level of insulin receptor activation can be based on established levels in healthy individuals. In a still further aspect, a standard activation level of insulin receptor activation can be based on established levels in the subject being treated prior to the determination of a need for increased insulin receptor activation.

For example, disclosed are methods of lowering blood sugar in a subject, the method comprising administering to the subject a therapeutically effective amount of a peptide comprising an insulin A chain peptide and an insulin B chain peptide, wherein the insulin B chain peptide comprises at least 32 amino acid residues, and wherein at least three of the amino acid residues of the insulin B chain peptide are lysine residue, thereby lowering blood sugar in the subject. In addition, disclosed are methods of lowering blood sugar in a subject, the method comprising administering to the subject a therapeutically effective amount of a peptide comprising an insulin A chain peptide and an insulin B chain peptide, wherein the peptide is directly conjugated to at least one organic borate group, thereby lowering blood sugar in the subject.

In various aspects, the insulin A chain peptide and the insulin B chain peptide are bonded via at least one disulfide bond, the insulin A chain peptide comprises the sequence of GIVEQCCHRICSLYQLENYCN (SEQ ID NO:1), and the insulin B chain peptide comprises the sequence of FVNQHLCGSHLVEALYLVCGERGFFYTPKTRKK (SEQ ID NO:8). In a further aspect, one or both of the B33 lysine residue and the B34 lysine residue are modified. In a still further aspect, one of the B33 lysine residue and the B34 lysine residue are modified. In yet a further aspect, the B33 lysine residue is modified. In an even further aspect, the B34 lysine residue is modified. In a still further aspect, both of the B33 lysine residue and the B34 lysine residue are modified.

In various aspects, the insulin A chain peptide comprises the sequence of GIVEQCCTSICSLYQLENYCG (SEQ ID NO:3), the insulin B chain peptide comprises the sequence of FVNQHLCGSHLVEALYLVCGERGFFYTPKTRKK (SEQ ID NO:8), the B29 lysine residue is not modified, each of the B33 lysine residue and the B34 lysine residue is directly conjugated to an organic borate group, and each occurrence of the organic borate group has a structure represented by a formula:

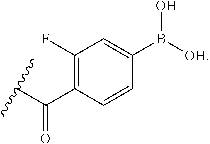 [SMILES: *C(=O)c1ccc(B(O)O)cc1F]

In various aspects, the subject is a mammal. In a further aspect, the mammal is a human.

In a further aspect, the subject has been diagnosed with a need for lowering blood sugar prior to the administering step. In a still further aspect, the method further comprises the step of identifying a subject in need of having their blood sugar lowered.

In a further aspect, the subject has been diagnosed with a disorder associated with high blood pressure such as, for example, diabetes and hyperglycemia. In a still further aspect, the method further comprises the step of identifying a subject in need of treatment of a disorder associated with high blood pressure such as, for example, diabetes and hyperglycemia.

In a further aspect, the subject has been diagnosed with a need for treatment of diabetes prior to the administering step. In a still further aspect, the method further comprises the step of identifying a subject in need of treatment of diabetes.

In various aspects, diabetes is type 1 diabetes. In a further aspect, diabetes is type 2 diabetes. In a still further aspect, diabetes is gestational diabetes.

### G. Methods of Using the Peptides

Herein, an improved insulin with increased solubility and elevated glucose concentrations (i.e., a smart insulin) is described. Without wishing to be bound by theory, two advantages of injecting a smart insulin, whose activity is modulated in vivo by circulating blood glucose levels, include: (1) errors in under-dosing insulin is markedly reduced because glucose-responsive insulin (GRI) derivatives are released from the subcutaneous depot whenever glucose levels are high; and (2) errors in overdosing insulin would be markedly reduced because GRI analogs would be inactivated when glucose levels start to decline, thus reducing the risk of hypoglycemia. Since higher amounts of glycated hemoglobin, a result of chronic hyperglycemia, are associated with complications such as cardiovascular diseases, nephropathy and retinopathy, the normoglycemia afforded by treatment with glucose-responsive insulin analogs have improved therapeutic value. GRI analogs can reduce the barrier of hypoglycemia for people with diabetes.

As disclosed, in one aspect, the smart insulin has similar activity with insulin glargine in high glucose conditions. In accordance with one aspect of the technology, smart insulin incorporates phenylboronic acids (PBA) on the insulin molecule. For example, a negatively-charged PBA-glucose complex can decrease the isoelectric point (pI) of insulin by binding to glucose, therefore increasing the solubility of insulin in high glucose concentrations to allow for more rapid entry into the bloodstream.

Thus, in various aspects, the peptides and pharmaceutical compositions of the invention are useful in treating or controlling diabetes. To treat or control the disorder, the peptides and pharmaceutical compositions comprising the peptides are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of diabetes.

The peptides or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of a disease or condition.

The therapeutically effective amount or dosage of the peptide can vary within wide limits. Such a dosage is adjusted to the individual requirements in each particular case including the specific peptide(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the peptide or composition to make up the daily dose. The dosage can be adjusted by the individual physician in the event of any contraindications. Dosage can vary, and can be administered in one or more dose administrations daily, for one or several days.

1. Treatment Methods

The peptides disclosed herein are useful for treating or controlling diabetes. Thus, provided is a method comprising administering a therapeutically effective amount of a composition comprising a disclosed compound to a subject.

a. Treating Diabetes

In one aspect, disclosed are methods of treating diabetes in a subject, the method comprising the step of administering to the subject an effective amount of at least one disclosed peptide.

Thus, in various aspects, disclosed are methods of treating diabetes in a subject, the method comprising administering to the subject a therapeutically effective amount of a peptide comprising an insulin A chain peptide and an insulin B chain peptide, wherein the insulin B chain peptide comprises at least 32 amino acid residues, and wherein at least three of the amino acid residues of the insulin B chain peptide are lysine residues, thereby treating diabetes in the subject.

In various aspects, disclosed are methods of treating diabetes in a subject, the method comprising administering to the subject a therapeutically effective amount of a peptide comprising an insulin A chain peptide and an insulin B chain peptide, wherein the peptide is directly conjugated to at least one organic borate group, thereby treating diabetes in the subject.

In a further aspect, the subject has been diagnosed with a need for treatment of diabetes prior to the administering step.

In a further aspect, the subject is a mammal. In a still further aspect, the mammal is a human.

In a further aspect, the method further comprises the step of identifying a subject in need of treatment of diabetes.

In a further aspect, the method further comprises the step of administering a therapeutically effective amount of at least one agent known to treat or control diabetes. Examples of agents known to treat or control diabetes include, but are not limited to, rapid-acting insulin, short-acting insulin, intermediate-acting insulin, long-acting insulin, metformin, an amylin analogue, and a GLP-1 receptor agonist (e.g., albiglutide, dulaglutide, exenatide, exenatide extended release, and liraglutide).

In a further aspect, the at least one compound and the at least one agent are administered sequentially. In a still further aspect, the at least one compound and the at least one agent are administered simultaneously.

In a further aspect, the at least one compound and the at least one agent are co-formulated. In a still further aspect, the at least one compound and the at least one agent are co-packaged.

In various aspects, diabetes is type 1 diabetes. In a further aspect, diabetes is type 2 diabetes. In a still further aspect, diabetes is gestational diabetes.

2. Use of Compounds

In one aspect, the invention relates to the use of a disclosed peptide or a product of a disclosed method. In a further aspect, a use relates to the manufacture of a medicament for the treatment of diabetes in a mammal.

Also provided are the uses of the disclosed peptides and products. In one aspect, the invention relates to use of at least one disclosed peptide. In a further aspect, the peptide used is a product of a disclosed method of making.

In a further aspect, the use relates to a process for preparing a pharmaceutical composition comprising a therapeutically effective amount of a disclosed peptide or a product of a disclosed method of making, for use as a medicament.

In a further aspect, the use relates to a process for preparing a pharmaceutical composition comprising a therapeutically effective amount of a disclosed peptide or a product of a disclosed method of making, wherein a pharmaceutically acceptable carrier is intimately mixed with a therapeutically effective amount of the peptide or the product of a disclosed method of making.

In various aspects, the use relates to a treatment of diabetes in a mammal. In one aspect, the use is characterized in that the mammal is a human. In one aspect, the use is characterized in that the diabetes is type 1 diabetes.

In a further aspect, the use relates to the manufacture of a medicament for the treatment of diabetes in a mammal.

It is understood that the disclosed uses can be employed in connection with the disclosed peptides, products of disclosed methods of making, methods, compositions, and kits. In a further aspect, the invention relates to the use of a disclosed peptide or a disclosed product in the manufacture of a medicament for the treatment of diabetes in a mammal.

3. Manufacture of a Medicament

In one aspect, the invention relates to a method for the manufacture of a medicament for treating diabetes in a mammal, the method comprising combining a therapeutically effective amount of a disclosed peptide or product of a disclosed method with a pharmaceutically acceptable carrier or diluent.

As regards these applications, the present method includes the administration to an animal, particularly a mammal, and more particularly a human, of a therapeutically effective amount of the peptide effective in the treatment of diabetes. The dose administered to an animal, particularly a human, in the context of the present invention should be sufficient to affect a therapeutic response in the animal over a reasonable timeframe. One skilled in the art will recognize that dosage will depend upon a variety of factors including the condition of the animal and the body weight of the animal.

The total amount of the peptide of the present disclosure administered in a typical treatment is preferably between about 10 mg/kg and about 1000 mg/kg of body weight for mice, and between about 100 mg/kg and about 500 mg/kg of body weight, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing, and frequency of administration, as well as the existence, nature, and extent of any adverse side effects that might accompany the administration of the peptide and the desired physiological effect. It will be appreciated by one of skill in the art that various conditions or disease states, in particular chronic conditions or disease states, may require prolonged treatment involving multiple administrations.

Thus, in one aspect, the invention relates to the manufacture of a medicament comprising combining a disclosed peptide or a product of a disclosed method of making, or a pharmaceutically acceptable salt, solvate, or polymorph thereof, with a pharmaceutically acceptable carrier or diluent.

4. Kits

In one aspect, disclosed are kits comprising a peptide comprising an insulin A chain peptide and an insulin B chain peptide, wherein the insulin B chain peptide comprises at least 32 amino acid residues, and wherein at least three of the amino acid residues of the insulin B chain peptide are lysine residues, and one or more of: (a) an agent known to treat diabetes; (b) instructions for administering the peptide for the treatment of diabetes; (b) instructions for treating diabetes; (c) instructions for lowering blood sugar.

In one aspect, disclosed are kits comprising a peptide comprising an insulin A chain peptide and an insulin B chain peptide, wherein the peptide is directly conjugated to at least one organic borate group, and one or more of: (a) an agent known to treat diabetes; (b) instructions for administering the peptide for the treatment of diabetes; (b) instructions for treating diabetes; (c) instructions for lowering blood sugar.

Examples of agents known to treat diabetes include, but are not limited to, agents known to increase insulin production, agents known to improve the body's use of insulin, and agents known to partially block the digestion of starches.

In a further aspect, the peptide and the agent are co-formulated. In a further aspect, the peptide and the agent are co-packaged.

In a further aspect, the peptide and the agent are administered sequentially. In a still further aspect, the peptide and the agent are administered simultaneously.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### G. Examples

Insulin glargine has two additional arginine residues in the C-terminus of the B chain, which leads to an increased isoelectric point (pI) and reduced solubility under physiological pH compared to native insulin. In an aspect, insulin derivatives are synthesized with both PBA- and positive-charged groups incorporated into insulin, retaining bioactivity. In accordance with the example, at low blood glucose conditions, these modified insulin molecules will remain mostly insoluble. However, as blood glucose levels rise, the equilibrium shifts, and free glucose will bind to PBA-forming negative charges, and reducing the pI. In a further aspect, incorporation of the PBA group itself reduces the solubility by 5-fold. In a still further aspect, other smart glargines with similar solubility profiles may be used. In yet a further aspect, different PBA groups may be used. Different PBA and amino acid combinations will result in different glucose-responsive properties and solubility. In an even further aspect, amino acid combinations such as Arg-Glu may be used to increase the solubility. Arg-Glu has a net charge of zero not affecting the pI. Furthermore, the polar functional groups from the side chain can increase the overall solubility. In a still further aspect, the PBA group (one or two) can be incorporated in B chain. In yet a further aspect, the solubility group is appended (Arg-Glu)n, n:1-3, in the C-terminal of A chain for simplicity. It is important to note other derivatives with similar initial solubility as glargine and with >5-fold solubility difference between 100 and 400 mg/dL glucose can be utilized.

In various aspects, synthesized analogs are tested in receptor activation assays identifying analogs that remain bioactive. In a further aspect, an insulin receptor (IR) binding assay using radiolabeled insulin is used to confirm the binding between analogs and IR. Insulin analogs with at least 85% potency in receptor activation and binding relative to insulin glargine can be selected for further characterization.

In various aspects, structural or sequence modification of an insulin molecule may result in altered binding affinities and activities to the insulin receptor (IR) and/or the insulin-like growth factor 1 receptor (IGF1R). Furthermore, activation of insulin signaling may also lead to a metabolic action (induce glucose uptake) and mitogenic action (growth and proliferation). In a further aspect, insulin derivatives may have an altered metabolic action and mitogenic action compared to human insulin. For example, insulin ×10 (B10 Asp human insulin) may induce cell proliferation in vitro and tumor formation in vivo.

Herein, the design and synthesis of “smart glargine” and its glucose-responsive properties is described. Smart glargine has a similar in vitro bioactivity as insulin glargine. It is further demonstrated that under varying glucose concentrations, smart glargine demonstrated a nearly 3-fold in vivo activity difference compared to insulin glargine and significantly reduced the incidence of hypoglycemia. Thus, without wishing to be bound by theory, smart glargine insulin represents a new design in achieving glucose-mediated control of insulin based on protein solubility.

The following preparations and examples are given to enable those skilled in the art to more clearly understand and to practice the present invention. They should not be considered as limiting the scope of the invention, but merely as being illustrative and representative.

### 1. General Information

All Fmoc-amino acids, reagents, and solvents were used without purification. Fmoc-amino acids, coupling reagents and 2-chlorotrityl chloride resin (cat. no. 03498) were purchased from Chem Impex International. Inc. Rink Amide MBHA resin HL (cat. no. 855118) and Novasyn TGA resin (cat. no. 855005) was obtained from Novabiochem and Rink amide ChemMatrix resin (cat. no. 7-600-1310) was purchased from Biotage. N,N′-dimethylforamide (DMF), dichloromethane (DCM), acetonitrile (ACN), methanol (MeOH), diethyl ether (Et2O), acetic acid (AcOH) and trifluoroacetic acid (TFA) were obtained from Fisher Scientific. Piperidine, triisopropylsilane (TIS), Hydroxybenzotriazole (HoBt), N,N′-diisopropylcarbodiimide (DIC), 2,2-dithiodipyridine (DTDP), 2,2-dithiobis(5-nitropyridine) (DTNP) were obtained from Sigma Aldrich. 1-[Bis(dimethylamino)methylene]-1H-1,2,3-triazolo[4,5-b]pyridinium3-oxid hexafluorophosphate (HATU) was purchased from Chem Impex International.Isoacyl-dipeptides Boc-Ser[Fmoc-Thr(tBu)]-OH were purchased from Novabiochem.

LC-MS system: Agilent 6120 Quadrupole LC/MS system on an XBridge C18 5-μm (50×2.1 mm) column with a linear gradient from 0 to 95% aqueous acetonitrile (0.1% formic acid) at 0.4 mL/min.

General RP-HPLC condition: All A-chains and AB chain dimer were purified by either a Phenomenax Luna C18 Column (5u, 100 Å, 250×21.2 mm) with a linear gradient from 15% to 50% aqueous acetonitrile (0.1% trifluoroacetic acid) over 60 min at a flow rate of 5 mL/min or a Phenomenax Jupitor C18 Column (5u, 300 Å, 250×10 mm) with a linear gradient from 15% to 50% aqueous acetonitrile (0.1% trifluoroacetic acid) over 40 min at a flow rate of 3 mL/min.

All B-chains were purified by similar methods with a different gradient from 30% to 65% aqueous acetonitrile (0.1% trifluoroacetic acid) over 60 min at a flow rate of 5 mL/min or over 40 min at a flow rate of 3 mL/min.

A chains were synthesized by a Biotage automated microwave peptide synthesizer (Initiator+ Alstra™) using Fmoc solid phase synthesis. Peptide synthesis was carried out on 0.1 mmol scale with a standard HATU/DIEA protocol. For Fmoc deprotection, 20% piperidine in DMF was added and mixed for 5 min twice at 25° C. For amino acid coupling, 0.2 M Fmoc-protected amino acid, 0.2 M HATU (coupling reagent), and 1.0 M DIEA (base) were prepared in DMF. In each cycle, 5 eq. amino acid, 5 eq. coupling reagent, and 10 eq. base was added into the reaction vessel and mixed for 5 min at 75° C. (for cysteine and histidine, mix for 10 min at 50° C.; for Arginine, mix for 15 min at 50° C. and couple twice). Upon completion of the peptide chain, resins were washed with DCM and dried using vacuum. Peptide was then cleaved by TFA, and further precipitated with cold ethyl ether, followed by HPLC purification and lyophilization.

B chains were synthesized by a Prelude X peptide synthesizer without heating. The synthesis protocol was the same as what is used for A chains except for the coupling time. For amino acid coupling, the reaction was mixed for 30 min at 25° C. with nitrogen bubbling. Detailed smart glargine synthetic protocols are described elsewhere herein.

### 2. Peptide Synthesis

The synthesis of A chain (InsA(G)) was conducted on 0.1 mmol Rink amide ChemMatrix resin (0.54 mmol/g) using a Biotage automated microwave peptide synthesizer. The C-terminal amino acid Asn was linked to the resin as Fmoc-Asp-OtBu through its side chain carboxyl group. Isoacyl-dipeptide Boc-Ser[Fmoc-Thr(tBu)]-OH was used as a single residue and coupled by a standard protocol as others. The peptide bound resin was treated with fresh 25% β-mercaptoethanol (15 mL) for 1.5 h twice and thoroughly washed with DMF (15 mL×3) and DCM (15 mL×3). Then DTNP (310 mg, 10 eq.) in 10 mL DCM was added to the resin and shaken for 1 h. The resin was washed again with DMF (15 mL×3) and DCM (15 mL×3) and treated with 1% TFA, 5% TIS in DCM (10 mL) for 2 min 5 times. Finally the resin was shaken in DCM (10 mL) for 1 h before it was cleaved by TFA/TIS/H2O (9 mL/500 μL/500 μL) for 2 h.

The synthesis of B chain R2 was conducted on 0.1 mmol 2-chlorotrityl chloride resin (0.4 mmol/g) using a Prelude X peptide synthesizer without heating. The C-terminal Arg was loaded to the resin using Fmoc-Arg(pbf)-OH/DIEA (4/4 eq. as to the resin) manually. The chain assembly was carried out as described in the general information section.

The synthesis of B chain RP2 was conducted on 0.1 mmol 2-chlorotrityl chloride resin (0.4 mmol/g) using a Prelude X peptide synthesizer without heating. The chain assembly was carried out as described in the general information section. The phenylboronic acid was installed via Dde-protected Lys. It was coupled as a standard amino acid. The N-terminal amino acid Phe was coupled using Boc-Phe-OH. After synthesis, the peptide bound resin was treated with 2% hydrazine for 5 min twice to remove the Dde group on Lys. After washing with DMF and DCM, phenylboronic acid was installed to lys using 4-carboxyphenylboronic acid (332 mg, 20 eq.), HoBt (270 mg, 20 eq.) and DIC (313 ul, 20 eq.) for 12 h or 4-carboxyphenylboronic acid (66 mg, 4 eq.), HATU (152 mg, 4 eq.) and DIEA (70 ul, 4 eq.) for 45 min twice. Finally, resin was cleaved by TFA/TIS/H2O (4.5 mL/250 μL/250 μL) with DTDP (330 mg, 15 eq.) for 2 h. The synthesis of B chain RF2 was synthesized in the same manner except the use of 4-carboxy-3-fluorophenylboronic acid.

### 3. Preparation of Insulin Analogs

The insulin analogs were prepared by combining A and B chain using a two-step method. A chain (4 mg, 1.63 μmol) and B chain (7.2 mg, 1.62 μmol) were mixed in the chain ligation buffer (6 M urea, 0.2 M NH₄OAc, pH 4.5, 0.8 mL). The combination reaction was allowed to proceed for 4 h at 25° C. before it was purified by RP-HPLC. Then the pooled fraction was adjusted to pH 8 by 1 M NH₄HCO₃ and lyophilized.

The lyophilized powder (10 mg, 1.47 μmol) was dissolved in a mixture solvent of AcOH (200 μL) and H₂O (800 μL) at 25° C., and treated with a freshly prepared iodine (11.2 mg, 44.1 μmol) solution in AcOH (3 mL) for 10 min with gentle agitation.

The oxidation was quenched by the addition of 1 M ascorbic acid until the iodine color (purple) disappeared. The final solution was diluted by H₂O (16 mL) and purified as described in the general information section.

### 4. In Vitro Receptor Binding Assay

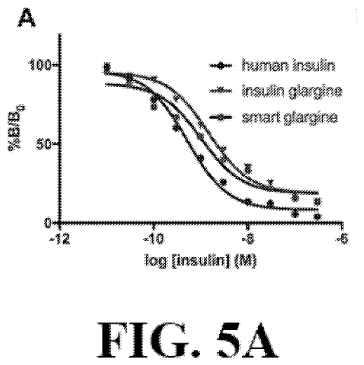

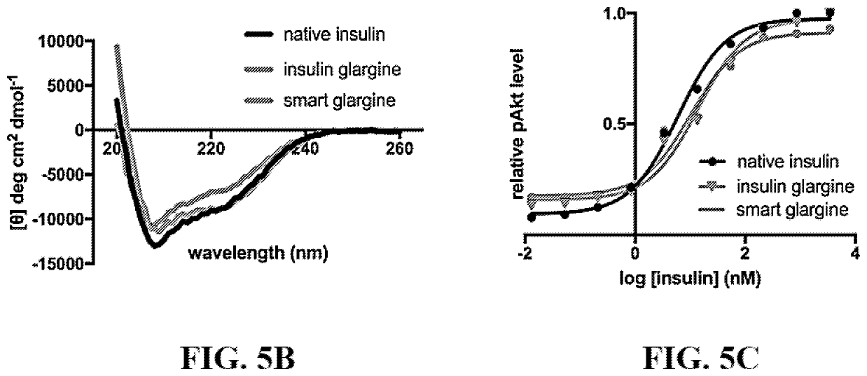

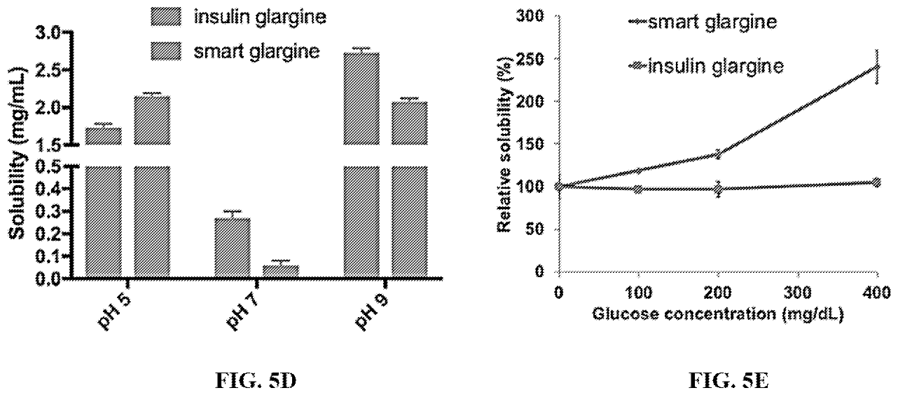

IR (isoform B) ectodomain with His-tag was immobilized in 96-well plates. Instead of using radiolabeled insulin, Eu-modified insulin was used. Time-resolved fluorescence was measured with 340-nm excitation and 612-nm emission filters. This assay was recently reported (Menting et al. (2016) Nat Struct Mol Biol). Using the binding assay, smart glargine was found to have a ˜2-fold reduction in binding affinity compared to native insulin. This is consistent with literature data about insulin glargine IR affinity (FIG. 5A).

### 5. In Vitro Bioactivity Assay

To measure the bioactivity of human insulin, insulin glargine, and smart glargine, pAkt Ser473 levels were measured in a mouse fibroblast cell line, NIH 3T3, overexpressing human IR-B (a gift from A. Morrione, Thomas Jefferson University). The cells were authenticated by western blotting to assess their level of IR expression compared with that of parent 3T3 cells: the NIH 3T3 cells showed an approximately ten-fold-higher level of expression than that of the parent. The NIH 3T3 cell line was cultured in DMEM (Thermo Fisher Scientific) with 10% FBS, 100 U/mL penicillin-streptomycin (Thermo Fisher Scientific) and 2 μg/mL puromycin (Thermo Fisher Scientific) and were shown to be free of mycoplasma contamination. For the assay, 40,000 cells per well were plated in 96-well plates with culture medium containing 1% FBS. 24 h later, 50 μL of insulin solution was pipetted into each well after the removal of the original medium. After a 30-min treatment, the insulin solution was removed, and a HTRF pAkt Ser473 kit (Cisbio, 64AKSPEH) was used to measure the intracellular level of pAkt Ser473.

Briefly, the cells were first treated with cell lysis buffer (50 μL per well) for 1 h under mild shaking. 16 μL of cell lysate was then added to 4 μL of detecting reagent in a white 384-well plate. After a 4-h incubation, the plate was read in a Synergy Neo plate reader (BioTek), and the data were processed according to the manufacturer's protocol. The assays were repeated a total of four times (biological replicates). Mean EC₅₀ values and their 95% confidence intervals were calculated (using Prism 8) after curve fitting with a nonlinear regression (one-site) analysis.

### 6. Additional Prophetic Bioactivity Assays

Selected insulin derivatives will be further tested for their effects on other key proteins in the insulin signaling pathway in order to confirm full activation. First, phosphorylation of IR in will be measured using western blots to confirm receptor phosphorylation. Second, phosphorylation of insulin receptor substrate 1 (IRS-1) will be measured using western blots. Third, phosphorylation of glycogen synthase kinase 3 (GSK-3) will also be measured because a reduced pGSK-3 level is expected when IR is activated by insulin. The GRI derivatives will also be evaluated for insulin signaling activation in both C2C12 and HepG2 cell lines due to the well-known insulin actions on muscle and liver cells.

### 7. Circular Dichroism

All CD spectra were recorded on an AVIV Model 410 spectrophotometer (AVIV) in water in a 1 mm QS quartz cuvette (Starna) at 25° C. Wavelength scans were performed at 1-nm resolution with 1-s averaging time. Data from double scans were averaged, blank subtracted, and normalized to mean residue ellipticity by the following equation: [θ]=100×θ/C×1×(n−1), where C is concentration of protein in mM, 1 is path length in centimeters, and n is the number of residues in the protein. The concentrations of the protein samples used for CD experiments were 100 μM.

### 8. Solubility Determination

To an Eppendorf tube, 1 mg peptide was added and suspended in 100 μl PBS buffer (pH 5, 7, and 9) with various concentrations of glucose (0-400 mg/dl). The peptide was added in excess to make a saturated solution with incompletely dissolved peptides at the bottom. Samples were vortexed for 5 min and gently shaken for overnight. Then they were centrifuged at 12,000 rpm for 10 min. The concentrations of saturated peptide solutions were determined by Nanodrop based on absorbance at 280 nm and calculated extinction coefficient.

### 9. Animals

Male Sprague-Dawley rats (SASCO SD, Strain code: 400; Charles River Laboratories, Inc., Wilmington, Mass.) weighing 250-300 g were housed in polyacrylic cages and maintained under standard housing conditions (room temperature 22-24° C. with 12 h light/dark cycle) at the University of Utah. The animals had free access to food and water, and were acclimatized to handling for 1 week before experimental procedure. All procedures were performed in accordance with the United States National Institutes of Health (NIH) Guide for the Care and Use of Laboratory Animals and approved by the Institutional Animal Care and Use Committee (IACUC) of University of Utah.

### 10. Glargine Sample Preparation

Commercial Lantus (100 U/ml) was purified by HPLC to obtain insulin glargine. Both lyophilized insulin glargine and smart glargine were dissolved in (3.63 mg/ml) diluent buffer, pH 4 containing similar constituents as of commercial Lantus diluent (30 μg zinc, 2.7 mg m-cresol, 20 mg glycerol 85%, 20 μg polysorbate 20).

### 11. Vascular Surgery

Rat was anesthetized with an intraperitoneal injection of ketamine/xylazine (75 mg/kg ketamine with 5 mg/kg xylazine) and an incision was made on the midline of ventral side of neck to implant vascular catheters under aseptic conditions. A micro-renathane catheter (MRE 025, Braintree Scientific Inc., Braintree, Mass.) was inserted into the right jugular vein and another catheter (MRE 033) was implanted into the left carotid artery. To maintain patency, all catheters were filled with a 40% polyvinylpyrrolidone (Sigma, MO) in heparin (1,000 units/ml; USP) and tunneled subcutaneously to place at the back of the neck. The animals were then allowed to recover in their home cages before being placed to the animal facility.

### 12. Modified Euglycemic and Hyperglycemic Clamps

To evaluate the action of both smart and commercially available glargine insulin, modified euglycemic and hyperglycemic clamps were performed in nondiabetic and diabetic rats, respectively. In these modified glucose clamps, the absorption characteristic of the insulin was investigated following the administration of a single dose of subcutaneous injection (as opposed to an intravenous infusion, as would occur in traditional glucose clamps). For euglycemic clamps, one week after vascular surgery the nondiabetic control rats were fasted overnight and the arterial and venous catheters were exteriorized under isoflurane anesthesia and extended via connector for blood sampling and to attach to the infusion pump, respectively. After 90 minutes resting period, the basal glucose levels were measured from arterial blood samples obtained from awake, unrestrained rats using glucometer (Ascensia Contour BG monitors, Bayer HealthCare, IN). Following baseline blood glucose measurement, all rats were injected subcutaneously with either commercial glargine insulin (i.e. 0.5 mg/kg) or synthesized smart glargine insulin (0.5 mg/kg). Blood glucose was measured at 10 minute interval throughout the clamp and a constant variable intravenous infusion of dextrose (50% w/v) was used to maintain euglycemia (90-110 mg/dl) for 4 hours.

For the hyperglycemic clamps, four days following vascular surgery, rats were intraperitoneally injected with streptozotocin (STZ; 65 mg/kg) to induce diabetes. Diabetic rats were chosen for these studies as they had the advantage of increased blood and insterstitial glucose concentrations, ideal for investigating the effects of high glucose levels on insulin bioavailability. Three days after STZ injection and after an overnight fast, all diabetic rats were injected subcutaneously with either commercial glargine insulin (i.e., 0.5 mg/kg) or synthesized smart glargine insulin (0.5 mg/kg) and subjected to a similar clamp protocol except that the rats were clamped at hyperglycemic levels (˜400 mg/dl) for 4 hours.

### 13. Insulin Tolerance Test (ITT)

Insulin tolerance test (ITT) was performed on STZ diabetic rats following a 4-5 h fast. After obtaining baseline blood glucose levels, rats were injected subcutaneously with either commercial glargine insulin (1 mg/kg) or synthesized smart glargine insulin (1 mg/kg). Tail vein samples were obtained to assess blood glucose levels every 15 minutes over four hours using glucometer (Ascensia Contour BG monitors, Bayer HealthCare, IN).

### 14. Statistical Analysis

The results are represented as mean±standard error of the mean (SEM). Data were analyzed by student (unpaired) “t” test. Repeated measures ANOVA (two-way) was performed to analyze the data for glucose clamps and ITT over the period of 4 h. Post-hoc analyses were performed by Tukey's multiple comparison tests. A level of 5% probability was considered as statistically significant.

### 15. Development of Insulin Analogs

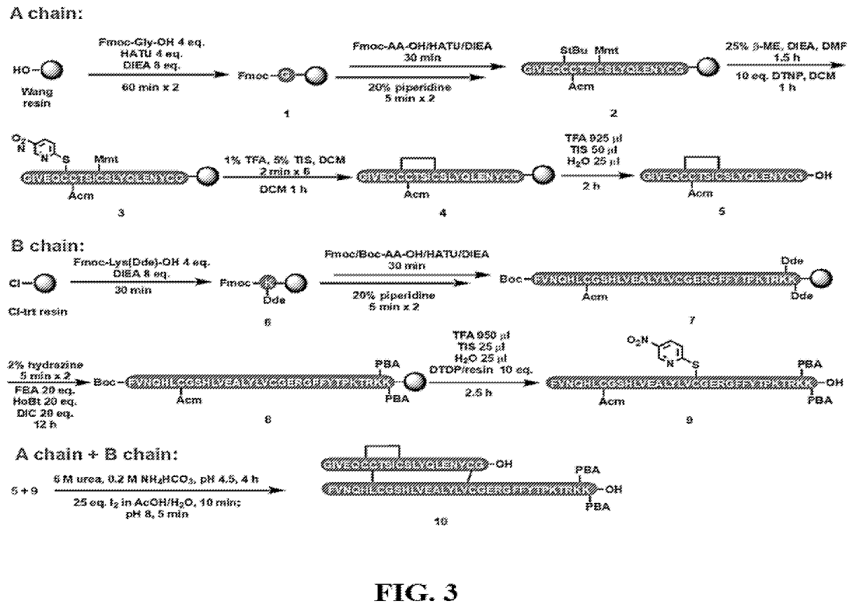

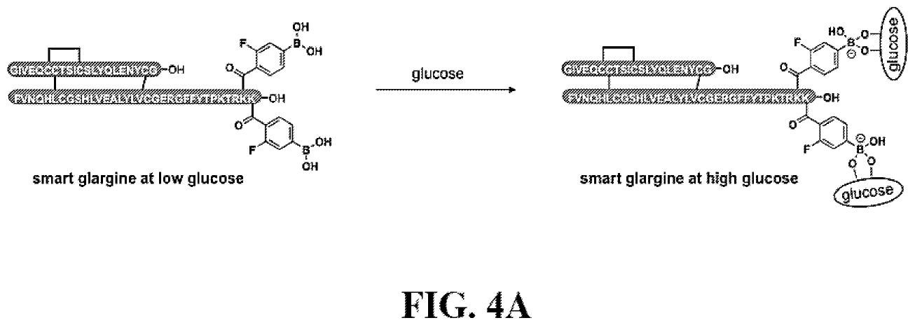

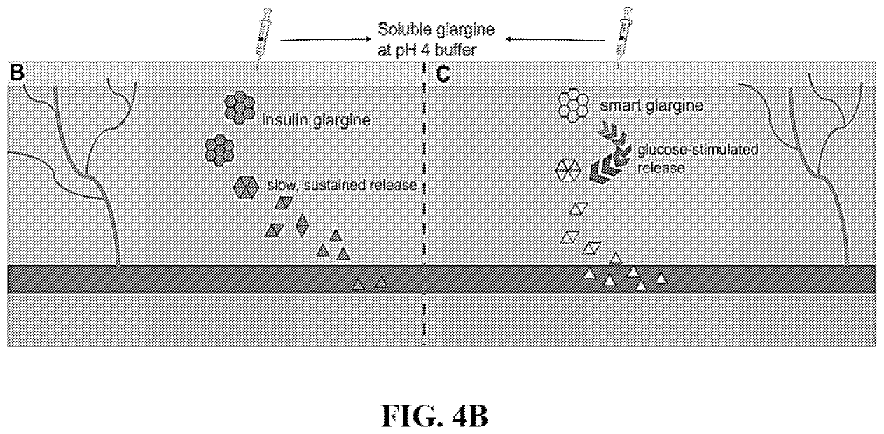

The chemical synthesis of smart glargine was achieved by using solid-phase peptide synthesis (FIG. 3). The A and B chains were synthesized separately. In order to avoid degradation of PBA under the harsh peptide coupling conditions, PBA was introduced at late stage after the whole B chain was synthesized utilizing Lys(Dde) residues. To form all three disulfide bonds in a controlled manner, four different Cys protecting groups were used as described in a previous report by Liu et al. (2014) Angewandte Chemie Int. Ed. 53(15): 3983-3987. First, A6 Cys(S-tBu) was deprotected using mercaptoethanol, followed by activation with 2,2′-dithiobis(5-nitropyridine) (DTNP). Next, All Cys(Mmt) was deprotected using 1% TFA to obtain the thiol. The A6-A11 intra-molecular disulfide bond was then formed through a disulfide substitution reaction. The A chain was then cleaved from the resin to give A7 Cys(Acm) and A20 free Cys (deprotection of Trt). The A and B chain were then ligated through a similar disulfide substitution reaction. The last disulfide bond formation was formed using iodine to obtain smart glargine after HPLC purification (>98% purity). 2-fluorophenylboronic acid was used due to its similar pKa to physiological pH (FIG. 4A) (Matsumoto et al. (2012) Angewandte Chemi 51(9): 2124-8).

Referring to FIG. 3, smart glargine was synthesized in two chains followed by chain combination using orthogonal protecting groups.

Referring to FIG. 4A, under high glucose conditions, the equilibrium shifts to negative-charged boronate complex upon glucose binding from the neutral boronic acid group. Referring to FIG. 4B, while insulin glargine has a slow and sustained release from subcutaneous depot, smart glargine was released in response to elevated glucose levels.

To determine whether PBA incorporation had an effect on secondary structure of insulin, the insulins were evaluated using near-UV circular dichroism (CD). All insulin molecules were observed to have CD spectra consistent primarily with a-helical secondary structure (FIG. 5B). To measure in vitro bioactivity, a cell-based insulin receptor activation assay was performed using pAkt level as indication of bioactivity (FIG. 5C). Both insulin glargine and smart glargine have a similar EC₅₀ (12 nM) for signal activation. Human native insulin has a 2-fold higher bioactivity than insulin glargine, which is consistent with literature reports (Varewijck and Janssen (2012) Endocrine-related cancer 19(5): F63-F75). Next, the solubility profile of both glargine molecules was measured. Both insulin glargine and smart glargine have a high solubility at pH=5 and pH=9, with a much lower solubility at pH=7, which is consistent with the biochemical design of insulin glargine (FIG. 5D). It was noted, however, that at pH=7, the solubility of smart glargine is lower than one-fourth that of insulin glargine (0.06 vs 0.28 mg/mL). Without wishing to be bound by theory, this is most likely due to the hydrophobic nature of PBAs. Next, the solubility profile was measured at pH=7 with various glucose concentrations (FIG. 5E). While insulin glargine has the same solubility from 0 to 400 mg/mL glucose, smart glargine has a ˜2.5-fold increased solubility over the same range. Without wishing to be bound by theory, this result supports the hypothesis that smart glargine has an increased solubility at high glucose conditions due to the negative charge from the boronate complex and the hydrophilic sugar attachment, thus demonstrating the biochemical basis of the glucose responsiveness.

Referring to FIG. 5B, circular dichroism spectra of human insulin, insulin glargine, and smart glargine is shown. Referring to FIG. 5C, in vitro activity of insulin analogs in activating insulin receptor using pAkt levels as a measurement is shown. The solubility profile of insulin glargine and smart glargine at pH=5, 7, and 9 is shown in FIG. 5D. The solubility profile of insulin glargine and smart glargine at pH=7 with glucose concentrations from 0 to 400 mg/dL is shown in FIG. 5E. While glargine has similar solubility in all conditions, smart glargine has elevated solubility in high glucose conditions (˜2-fold increase between 100 and 400 mg/dl).

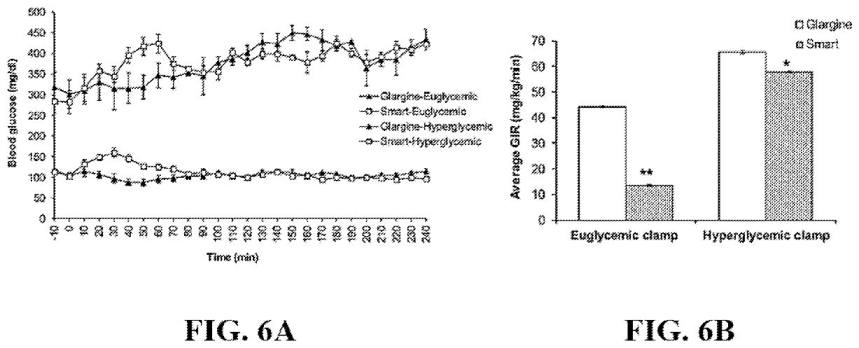

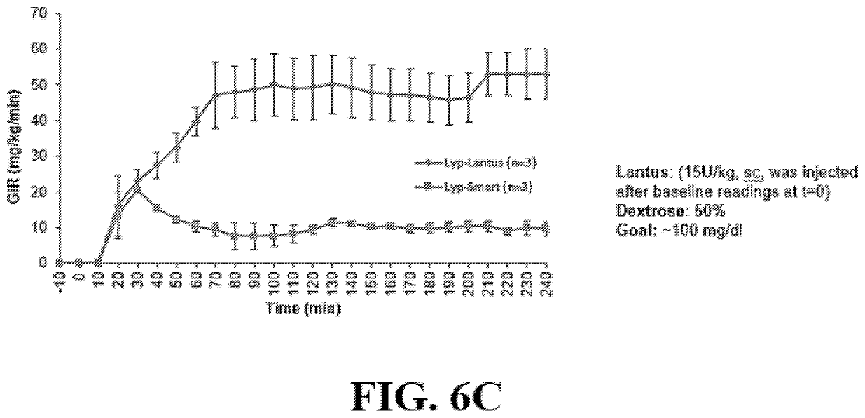

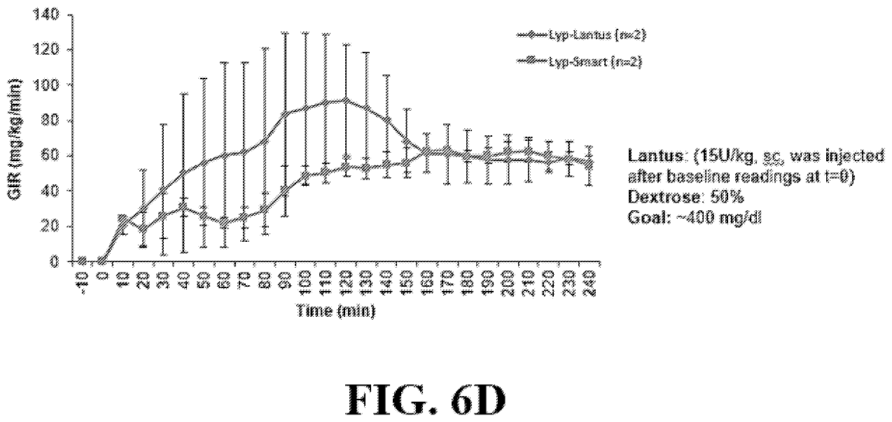

To establish the in vivo glucose responsiveness of smart glargine, euglycemic and hyperglycemic clamp studies were performed to compare and contrast the in vivo biological activities of both smart glargine and commercially available insulin glargine. After a subcutaneous injection of insulin (0.5 mg/kg), blood glucose levels were well matched (by experimental design) for both insulin glargine and smart glargine-treated rats during both the euglycemic and hyperglycemic clamp protocols (FIG. 6A). During the hyperglycemic clamp (˜400 mg/dL glucose), the glucose infusion rate needed to maintain hyperglycemia for the smart glargine-treated rats was 88% that of the insulin glargine-treated rats (FIG. 6B). However, during the euglycemic clamp (˜100 mg/dL glucose), the rate of exogenous glucose infusion in the smart glargine-treated rats was markedly reduced (to about 30% that of the insulin glargine-treated rats) (FIG. 6B) and this difference was highly significant (P<0.01). Without wishing to be bound by theory, this result indicates that smart glargine has similar in vivo bioactivity as insulin glargine under hyperglycemic conditions, but has greatly reduced activity under euglycemic conditions. This 2.9-fold difference in relative bioactivity demonstrates the glucose responsiveness of smart glargine in vivo.

Referring to FIG. 6A, blood glucose levels (mg/dl) during euglycemic (˜100 mg/dl) and hyperglycemic (˜400 mg/dl) clamps performed in nondiabetic control and streptozotocin (STZ)-diabetic rats, respectively, are shown. In both euglycemic and hyperglycemic clamps, rats were injected subcutaneously with either insulin glargine (0.5 mg/kg) or smart glargine (0.5 mg/kg) after baseline readings at t=0. Data are expressed as mean±SEM (n=5-6/group). Repeated measures ANOVA (two-way) followed by post-hoc test with Tukey's comparisons. Dextrose: 50%.

Referring to FIG. 6B, average glucose infusion rate (m per kg*min) during the last hour of euglycemic (˜100 mg/dl) and hyperglycemic (˜400 mg/dl) clamps is shown. In both euglycemic and hyperglycemic clamps, rats were injected with either insulin glargine (0.5 mg/kg) or smart glargine (0.5 mg/kg) subcutaneously. Data are expressed as mean±SEM (n=5-6/group). *P<0.05, **P<0.01 Vs Glargine; Student “t” (unpaired) test.

Referring to FIG. 6C and FIG. 6D, GIR (mg/kg/min) during euglycemic (FIG. 6C, ˜100 mg/dl) and hyperglycemic (FIG. 6D, ˜400 mg/dl) clamps performed in nondiabetic control and streptozotocin (STZ)-diabetic rats, respectively, are shown. In both euglycemic and hyperglycemic clamps, rats were injected subcutaneously with either insulin glargine (0.5 mg/kg) or smart glargine (0.5 mg/kg) after baseline readings at t=0. Dextrose: 50%.

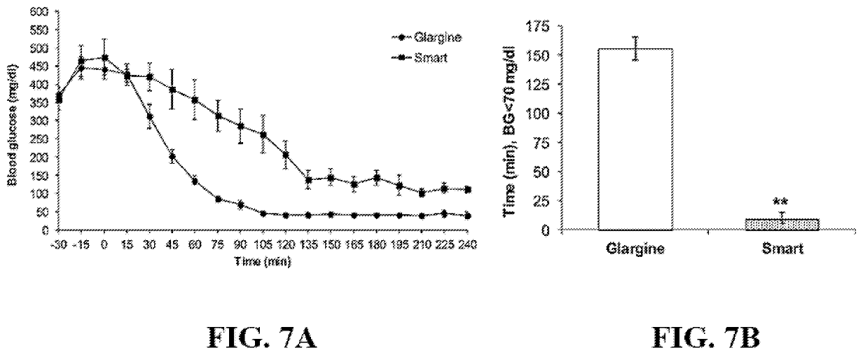

From the results of the clamp studies, it was hypothesized that smart glargine may be less likely to cause hypoglycemia. To evaluate the potential for insulin-induced hypoglycemia, high dose (1 mg/kg) insulin tolerance tests (ITTs) were performed in STZ-induced diabetic rats. In the absence of glycemic clamp conditions, the subcutaneous administration of both insulin glargine and smart glargine (1 mg/kg) lowered blood glucose levels (FIG. 7A). The nadir blood glucose levels reached in insulin glargine-treated rats was ˜40 mg/dl. Evidence of ongoing insulin absorption/action was noted by the maintenance of hypoglycemia for greater than 2 hours. This persistent insulin action is particularly impressive because it is maintained in the setting of a (likely) counter-regulatory response to hypoglycemia. Conversely, an equal dose of smart glargine resulted in a more gradual lowering of blood glucose and a nadir blood glucose level of ˜102 mg/dl. To quantify the hypoglycemic potency of these insulins, the duration of time during which the blood glucose remained hypoglycemic (<70 mg/dl) was quantified. Smart glargine-treated rats remained hypoglycemic for a significantly shorter duration as compared to commercial glargine-treated rats (FIG. 7B). Without wishing to be bound by theory, this 15-fold less hypoglycemic potency demonstrates that smart glargine portends a reduced risk of causing hypoglycemia as compared to insulin glargine.

Referring to FIG. 7A, blood glucose levels (mg/dl) during insulin tolerance tests (ITTs) performed in STZ-diabetic rats are shown. After obtaining baseline blood glucose readings, rats were injected with either insulin glargine (1 mg/kg) or smart glargine (1 mg/kg) subcutaneously. Data are expressed as mean±SEM (n=5-6/group). Repeated measures ANOVA (two-way) followed by post-hoc test with Tukey's comparisons.

Referring to FIG. 7B, time (min) during which the blood glucose levels remained below 70 mg/dl during the ITT in rats injected with either insulin glargine (1 mg/kg) or smart glargine (1 mg/kg) is shown. Data are expressed as mean±SEM (n=5-6/group). **P<0.01 vs Glargine; Student “t” (unpaired) test.

This study was undertaken with a goal to increase the therapeutic index of insulin. The innovation was based on the use of phenylboronic acids to convert insulin glargine into smart glargine allowing for glucose-dependent solubility at physiological pH. Unlike the steady release of commercially available insulin glargine into the bloodstream from the subcutaneous depot; the in vivo bioactivity profiles (FIG. 7B) would suggest that smart glargine exhibits relatively high absorption under high glucose conditions and markedly less absorption under lower glucose conditions. Interestingly, a lesser rate of insulin absorption from the subcutaneous depot (under euglycemic conditions) would give smart glargine the theoretical advantage of prolonging the duration of insulin action. Another unique advantage of this GRI derivative is its compatibility with other published GRI designs. For example, smart glargine can potentially be modified to create a smart glargine-carbohydrate conjugate based on the recent Merck strategies targeting mannose receptors (Kaarsholm et al. (2018) Diabetes 67(2): 299-308; Yang et al. (2018) JCI Insight 3(1)). Since the two designs have completely different mechanisms of action, such a combination has the potential to further enhance glucose responsiveness.

Insulin-induced hypoglycemia is the most serious acute complication of insulin therapy. Although the introduction of fast- and long-acting insulin analogs have led to reduced risk of hypoglycemia as compared to native insulins, these insulin analogs still have narrow therapeutic windows because once injected, insulin absorption into the bloodstream is continuous and independent of ambient glucose concentrations (Berenson et al. (2011) Annals of the New York Academy of Sciences 1243: E40-E54) (FIG. 4B). Consistent with this notion of persistent insulin action, it is noted that pharmacological dose of commercially available insulin glargine caused prolonged hypoglycemia (FIG. 7B). However, an equimolar dose of glucose responsive smart glargine demonstrated less hypoglycemia potency, as noted by: (1) a lowering of blood glucose level to a nadir of 102 mg/dl; and (2) a 15-fold reduced-duration of hypoglycemia. Without wishing to be bound by theory, several factors may have contributed to this beneficial finding. First, independent of relative bioactivity measurements ascertained during the steady-state portion of the glycemic clamp experiments, the more gradual decline in blood glucose levels during the insulin tolerance test with smart glargine (as compared to insulin glargine) indicates slower absorption following subcutaneous injection (FIG. 7A). This decreased rate of absorption of smart glargine at a physiological pH is consistent with the in vitro findings (FIG. 5D) and likely represents the hydrophobic nature of PBAs. Second, a 12% lower bioactivity for smart glargine under hyperglycemic conditions may have made a minor contribution to this observed effect. Third, as blood glucose approached euglycemia, it is proposed that the markedly reduced solubility of smart glargine at euglycemia (FIG. 5E and FIG. 7B) prevented the development of hypoglycemia. Of particular note, it is proposed that this slower onset and lower in vivo activity of smart glargine is not due to a reduction in intrinsic insulin-stimulatory activity because smart glargine and commercially available insulin glargine demonstrated identical bioactivity in activating insulin receptor signaling in vitro (FIG. 5C). In summary, smart glargine demonstrated an overall decrease in solubility, as noted in vitro (FIG. 5D) and also noted by slower onset of action noted in vivo (FIG. 7A); yet smart insulin did demonstrate an increased relative solubility under high glucose conditions in vitro (FIG. 5E) and in vivo (FIG. 6B), thus demonstrating glucose-responsive properties. Overall, these novel findings indicate that it is possible to manipulate the bioactivity profile of insulin by altering the overall solubility of glargine in a glucose-responsive fashion.

In summary, evidence for the synthesis of a glucose-responsive smart glargine is provided. It is superior in preventing hypoglycemia as compared to insulin glargine is presented. Rapid clinical development of insulin derivatives with glucose-responsive properties can help people with insulin-treated diabetes achieve glycemia goals while minimizing the risk for hypoglycemia.

### 16. Prophetic Example: Design and Chemical Synthesis of Glucose Responsive Insulin (GRI) Derivatives to Maximize Glucose Responsiveness

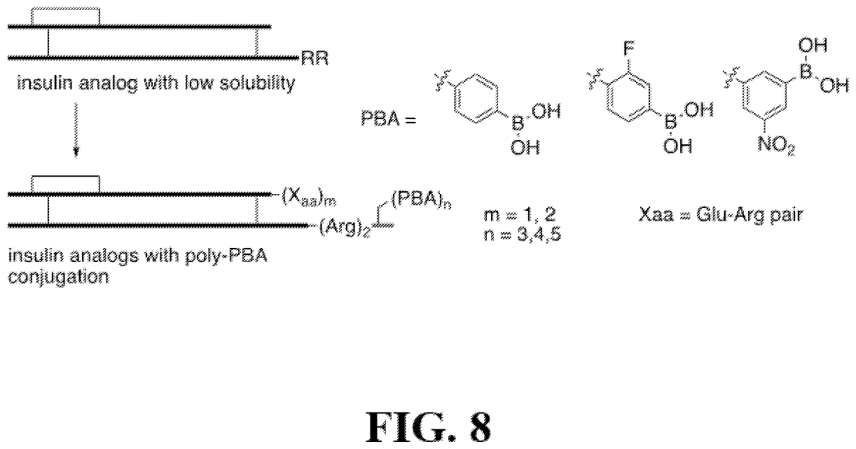

Insulin glargine has two additional arginine residues in the C-terminus of the B chain, which leads to an increased pI and reduced solubility under physiological pH compared to native insulin. Here, the goal is to synthesize insulin derivatives with both PBA and positively-charged groups incorporated into the insulin, while retaining bioactivity. At low blood glucose conditions, these modified insulin molecules will remain mostly insoluble. However, as blood glucose levels rise, the equilibrium shifts, and free glucose will bind to PBA forming negative charges, and reducing the pI (see FIG. 4B). In order to fully utilize the long-acting properties of glargine and maximize the glucose responsiveness, insulin derivatives with glucose-sensing PBA groups will be synthesized, while maintaining relatively low solubility at pH 7. In this case, different PBA and amino acid combinations will result in different glucose-responsive properties and solubility (PBA is aromatic and hydrophobic and, therefore, will lead to reduced baseline solubility). The baseline solubility of smart glargine is already 4-fold lower than that of insulin glargine. To overcome this limitation, amino acid combinations such as Arg-Glu will be explored to increase the solubility. Arg-Glu has a net charge of zero so it will not affect the pI. Furthermore, the polar functional groups from the side chain would increase the overall solubility. Since the PBA groups (3,4,5) will be incorporated in the B chain, the solubility group (Arg-Glu)n, n=1-2, will be incorporated in the C-terminal of A chain (FIG. 8). A variety of PBAs will be assessed for glucose-sensing properties in the context of enhancing glucose responsiveness. Because glucose binding is highly dependent on the pKₐ of the PBA, PBAs with pKₐ ranging from 7.0 to 7.8 will be used (FIG. 8). Therefore, a total of 18 insulin derivatives will be synthesized.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

1. A peptide comprising an insulin A chain peptide and an insulin B chain peptide, wherein the insulin B chain peptide comprises at least 32 amino acid residues, and wherein at least three of the amino acid residues of the insulin B chain peptide are lysine residues.

2. The peptide of claim 1, wherein the insulin A chain peptide is at least 70% identical to wild type human insulin A chain peptide.

3. The peptide of claim 1, wherein the insulin A chain peptide comprises the sequence of GIVEQCCTSICSLYQLENYCN (SEQ ID NO:1).

4. The peptide of claim 1, wherein the insulin A chain peptide comprises the sequence of GIVEQCCTSICSLYQLENYCG (SEQ ID NO:3).

5. The peptide of claim 1, wherein the insulin B chain peptide comprises at least 34 amino acid residues.

6. The peptide of claim 1, wherein an amino acid at position B29 is a lysine residue.

7. (canceled)

8. The peptide of claim 1, wherein an amino acid at position B33 is a lysine residue.

9. (canceled)

10. The peptide of claim 1, wherein an amino acid at position B34 is a lysine residue.

11. (canceled)

12. The peptide of claim 1, wherein each of an amino acid at position B29, an amino acid at position B33, and an amino acid at position B34 are lysine residues.

13. (canceled)

14. The peptide of claim 1, wherein the insulin B chain peptide comprises the sequence of FVNQHLCGSHLVEALYLVCGERGFFYTPKT (SEQ ID NO:2).

15. The peptide of claim 1, wherein the insulin B chain peptide comprises the sequence of FVNQHLCGSHLVEALYLVCGERGFFYTPKTR (SEQ ID NO:4), FVNQHLCGSHLVEALYLVCGERGFFYTPKTRR (SEQ ID NO:5), or FVNQHLCGSHLVEALYLVCGERGFFYTPKTRRR (SEQ ID NO:6).

16. The peptide of claim 1, wherein the insulin B chain peptide comprises the sequence of FVNQHLCGSHLVEALYLVCGERGFFYTPKTRKK (SEQ ID NO:8).

17. The peptide of claim 1, wherein the insulin A chain peptide and the insulin B chain peptide are bonded via at least one disulfide bond.

18. The peptide of claim 1, wherein at least two of the lysine residues are on the insulin B chain peptide's C-terminus.

19. The peptide of claim 1, wherein the peptide is a monomer.

20. The peptide of claim 1, wherein the insulin A chain peptide and the insulin B chain peptide are bonded via at least one disulfide bond, wherein the insulin A chain peptide comprises the sequence of GIVEQCCHRICSLYQLENYCN (SEQ ID NO:1), and wherein the insulin B chain peptide comprises the sequence of FVNQHLCGSHLVEALYLVCGERGFFYTPKTRKK (SEQ ID NO:8).

21. The peptide of claim 20, wherein one or both of the B33 lysine residue and the B34 lysine residue are modified.

22-38. (canceled)

39. A pharmaceutical composition comprising the peptide of claim 1, and a pharmaceutically acceptable carrier.

40. A method of treating diabetes in a subject, the method comprising administering to the subject a therapeutically effective amount of the peptide of claim 1, thereby treating diabetes in the subject.

41-51. (canceled)

52. A method of lowering blood sugar in a subject, the method comprising administering to the subject a therapeutically effective amount of the peptide of claim 1, thereby lowering blood sugar in the subject.

53-57. (canceled)
